(19) United States
(12) Patent Application Publication
(10) Pub. No.: US 2016/0270676 A1
(43) Pub. Date: Sep. 22, 2016

(54) BIOLOGICAL INFORMATION DETECTION SENSOR AND BIOLOGICAL INFORMATION DETECTION DEVICE

(71) Applicant: Seiko Epson Corporation, Tokyo (JP)

(72) Inventor: Hideto Yamashita, Suwa-shi (JP)

(21) Appl. No.: 15/004,309

(22) Filed: Jan. 22, 2016

(30) Foreign Application Priority Data
Mar. 19, 2015 (JP) 2015-056434

(51) Int. Cl. A61B 5/024

(52) U.S. Cl. CPC A61B 5/02427; A61B 5/02438; A61B 2562/0219; A61B 2562/0238

(57) ABSTRACT

A biological information detection sensor includes a first light emitter configured to emit light to a detection part, a first light receiver and a second light receiver configured to receive the light reflected in the detection part, a light blocker disposed between the first light emitter and the first and second light receivers and configured to block the light made directly incident on the first light receiver and the second light receiver from the first light emitter, a supporting body on which the first light emitter, the first light receiver, the second light receiver, and the light blocker are disposed, and a controller configured to analyze biological information on the basis of light reception result received from the first light receiver and the second light receiver.

### CROSS-REFERENCES TO RELATED APPLICATIONS

This application claims priority to Japanese Patent Application No. 2015-056434, filed Mar. 19, 2015, the entirety of which is herein incorporated by reference.

### BACKGROUND

1. Technical Field

The present invention relates to a biological information detection sensor and a biological information detection device.

2. Related Art

There has been proposed a pulse-rate measuring device that measures a pulse wave in a wrist or the like of a subject and measures a pulse rate (see, JP-A-2007-75482 (Patent Literature 1)).

The pulse-rate measuring device described in Patent Literature 1 includes a thin film disposed on the skin of the subject and one optical sensor including a light emitting element and a light receiving element. Light emitted from the light emitting element is emitted toward the thin film. The light reflected by the thin film is received by the light receiving element. The position of the thin film changes in synchronization with a beat of the subject. Therefore, a reflecting direction of the light emitted from the light emitting element and reflected by the thin film changes according to the change of the position. The intensity of the light made incident on the light receiving element changes. That is, the pulse-rate measuring device described in Patent Literature 1 outputs, as a pulse wave signal, a luminance change of the reflected light received by the light receiving element.

Incidentally, the pulse wave signal is a signal corresponding to the pulsation of a blood vessel. The luminance change of the reflected light by, other than the pulse, for example, movement of the blood vessel due to a body motion occurs. If a noise component due to such a body motion is included in a detection signal by the light receiving element, it is likely that the pulse rate cannot be accurately measured. Therefore, by removing the body motion noise component from the detected pulse wave signal to obtain a pulse signal, it is possible to accurately calculate and measure the pulse rate on the basis of the pulse signal.

However, in the biological information measuring device described in Patent Literature 1, the optical sensor includes one light emitting element and one light receiving element. Therefore, there is a problem in that the noise due to the body motion cannot be sufficiently reduced.

Therefore, there is a demand for a configuration capable of improving detection accuracy of a pulse wave and accurately measure a pulse rate.

### SUMMARY

An advantage of some aspects of the invention is to provide a biological information detection sensor and a biological information detection device that can accurately detect biological information.

A biological information detection sensor according to a first aspect of the invention includes: a first light emitter configured to emit light to a detection part; a first light receiver and a second light receiver configured to receive the light reflected in the detection part; a light blocker disposed between the first light emitter and the first and second light receivers and configured to block the light made directly incident on the first light receiver and the second light receiver from the first light emitter; a supporting body on which the first light emitter, the first light receiver, the second light receiver, and the light blocker are disposed; and a controller configured to analyze biological information on the basis of light reception result received from the first light receiver and the second light receiver.

Note that, as the biological information, a waveform of a pulse and a pulse rate (a pulse rate per unit time) can be illustrated. As the supporting body, a substrate mountable with a circuit element and the like can be illustrated.

According to the first aspect, since the light blocker is disposed between the first light emitter and the first and second light receivers, it is possible to suppress the light emitted from the first light emitter from being made directly incident on and received by the first and second light receivers. The controller can analyze the biological information on the basis of light reception results by the first and second light receivers of the light emitted from the first light emitter and reflected in the detection part. Therefore, compared with a biological information measuring device in which only one light receiving element is provided like the biological information measuring device described in Patent Literature 1, it is possible to accurately detect biological information.

In the first aspect, it is preferable that the light blocker configures at least a part of a frame that surrounds the first light receiver and the second light receiver viewed from the detection part side.

According to the first aspect with this configuration, since the first and second light receivers are surrounded by the frame, it is possible to further reduce the possibility that the light emitted from the first light emitter is made directly incident on and received by the first and second light receivers. It is possible to suppress external light deriving from a portion other than the light emitter from being made incident on the first and second light receivers. Therefore, the first and second light receivers can surely receive the light made incident via the detection part. Therefore, it is possible to more accurately detect biological information.

In the first aspect, it is preferable that the first light emitter and the first and second light receivers are disposed to differentiate the distance between the first light emitter and the first light receiver and the distance between the first light emitter and the second light receiver.

According to the first aspect with this configuration, it is possible to differentiate, with the first light receiver and the second light receiver, light reception results of the light made incident via the detection part from each other. In other words, the first light receiver and the second light receiver can receive lights having different kinds of information. Consequently, the controller can analyze the biological information on the basis of the light reception results received from the first light receiver and the second light receiver. Therefore, it is possible to further improve detection and analysis accuracy of biological information.

In the first aspect, it is preferable that a light receiving surface of the first light receiver and a light receiving surface of the second light receiver are respectively disposed in positions at different distances from the supporting body.

Note that, as the light receiving surfaces, besides photosensitive regions, that is, impurity regions of the first light receiver and the second light receiver, when the first light receiver and the second light receiver are covered with a transparent member or the like, an upper surface of the transparent member can be illustrated.

According to the first aspect with this configuration, it is possible to differentiate the lengths of the optical paths or the routes of the light emitted from the first light emitter and made incident on the first light receiver and the second light receiver via the detection part. In other words, the first light receiver and the second light receiver can receive lights having different kinds of information. Therefore, since it is possible to differentiate the light reception results by the first light receiver and the second light receiver, it is possible to improve the detection and analysis accuracy of biological information.

In the first aspect, it is preferable that the upper surface of the first light receiver and the upper surface of the second light receiver are respectively disposed in positions at different distances from the supporting body.

According to the first aspect with this configuration, it is possible to differentiate the lengths of the optical paths or the routes of the light emitted from the first light emitter and made incident on the first light receiver and the second light receiver via the detection part. In other words, the first light receiver and the second light receiver can receive lights having different kinds of information. Therefore, since it is possible to differentiate the light reception results by the first light receiver and the second light receiver, it is possible to improve the detection and analysis accuracy of biological information.

In the first aspect, it is preferable that a sectional shape including the center of the first light receiver and perpendicular to the light blocker and a sectional shape including the center of the second light receiver and perpendicular to the light blocker are different.

Note that, as an example of the different sectional shapes, different height dimensions, different sectional shapes, and different disposition positions of the first light receiver and the second light receiver can be illustrated besides different thickness dimensions of the frame.

According to the first aspect with this configuration, as in the case in which the distances to the first light receiver and the second light receiver from the supporting body are different, it is possible to differentiate the lengths of the optical paths or the routes of the light having different kinds of information emitted from the first light emitter and made incident on the first light receiver and the second light receiver via the detection part. In other words, the first light receiver and the second light receiver can receive the lights having the different kinds of information. Therefore, since it is possible to differentiate the light reception results by the first light receiver and the second light receiver, it is possible to surely improve the detection and analysis accuracy of biological information.

In the first aspect, it is preferable that the distance between the light blocker and the first light receiver and the distance between the light blocker and the second light receiver are different in plan view.

According to the first aspect with this configuration, as in the case in which the distances from the supporting body to the first light receiver and the second light receiver are different, it is possible to differentiate the lengths of the optical paths or the routes of the light having different kinds of information emitted from the first light emitter and made incident on the first light receiver and the second light receiver via the detection part. In other words, the first light receiver and the second light receiver can receive the lights having the different kinds of information. Therefore, since it is possible to differentiate the light reception results by the first light receiver and the second light receiver, it is possible to surely improve the detection and analysis accuracy of biological information.

In the first aspect, it is preferable that the distance from the supporting body to the upper surface of the light blocker is longer than the distance from the supporting body to the light receiving surface of the first light receiver and the light receiving surface of the second light receiver in plan view.

According to the first aspect with this configuration, since the distance from the supporting body to the upper surface of the light blocker is longer than the distance from the supporting body to the light receiving surfaces of the light receivers, it is possible to further reduce the possibility that the light emitted from the first light emitter is directly made incident on and received by the first and second light receivers. It is possible to further suppress the external light deriving from a portion other than the light emitter from being made incident on the first and second light receivers. Therefore, since the first and second light receivers can surely receive the light made incident via the detection part, it is possible to more accurately detect biological information.

In the first aspect, it is preferable that the biological information detection sensor further includes a second light emitter disposed on the supporting body and configured to emit light to the detection part, and the first light emitter and the second light emitter are disposed on the supporting body to be disposed in positions where at least one of the distances from the supporting body, the distances from the first light receiver, and the distances from the second light receiver are different.

According to the first aspect with this configuration, since the first light emitter and the second light emitter are respectively disposed in the positions, as explained above, it is possible to differentiate the lengths of the optical paths or the routes of the light emitted from the first light emitter and made incident on the first light receiver and the second light receiver via the detection part. Therefore, since it is possible to surely differentiate the light reception results by the first light receiver and the second light receiver, it is possible to surely improve the detection and analysis accuracy of biological information.

In the first aspect, it is preferable that the biological information detection sensor further includes a transparent member disposed around at least one of the first light receiver and the second light receiver and configured to differentiate, on the first light receiver side and the second light receiver side, pressing forces applied to the detection part.

According to the first aspect with this configuration, when the biological information detection sensor is worn on a user, the pressing force on the first light receiver side applied to the detection part and the pressing force on the second light receiver side applied to the detection part can be differentiated by a pressing force adjuster. Consequently, it is possible to differentiate SN ratios of electric signals generated by photoelectrically converting the light made incident on the first light receiver and the second light receiver because pressing states are different. By processing the electric signals, it is possible to reduce a noise component. It is possible to surely improve the detection and analysis accuracy of biological information.

A biological information detection device according to a second aspect of the invention includes the biological information detection sensor described above.

According to the second aspect, it is possible to achieve effects same as the effects of the biological information detection sensor according to the first aspect.

### BRIEF DESCRIPTION OF THE DRAWINGS

The invention will be described with reference to the accompanying drawings, wherein like numbers reference like elements.

FIG. 1 is a front view showing a biological information detection device according to a first embodiment of the invention.

FIG. 2 is a rear view showing the biological information detection device according to the first embodiment.

FIG. 3 is a side view showing the biological information detection device according to the first embodiment.

FIG. 4 is a plan view showing an example of the configuration of a detector in the first embodiment.

FIG. 5 is a sectional view showing an example of the configuration of the detector in the first embodiment.

FIG. 6 is a diagram for explaining noise reduction processing for a first detection signal based on a second detection signal performed using a spectral subtraction method in the first embodiment.

FIG. 7 is a diagram for explaining the noise reduction processing for the first detection signal based on the second detection signal performed using the spectral subtraction method in the first embodiment.

FIG. 8 is a diagram for explaining a flow of a processing signal in the first embodiment.

FIG. 9 is a sectional view showing an example of the configuration of a detector according to a modification of the first embodiment.

FIG. 10 is a sectional view showing an example of a detector of a biological information detection device according to a second embodiment of the invention.

FIG. 11 is a sectional view showing an example of the configuration of a detector according to a first modification of the second embodiment.

FIG. 12 is a sectional view showing an example of the configuration of a detector according to a second modification of the second embodiment.

FIG. 13 is a sectional view showing an example of the configuration of a detector according to a third modification of the second embodiment.

FIG. 14 is a sectional view showing an example of the configuration of a detector according to a fourth modification of the second embodiment.

FIG. 15 is a sectional view showing the configuration of a detector of a biological information detection device according to a third embodiment of the invention.

FIG. 16 is a plan view showing an example of the configuration of a detector according to a first modification of the third embodiment.

FIG. 17 is a plan view showing an example of the configuration of a detector according to a second modification of the third embodiment.

FIG. 18 is a plan view showing the configuration of a detector according to a third modification of the third embodiment.

FIG. 19 is a plan view showing the configuration of a detector of a biological information detection device according to a fourth embodiment of the invention.

FIG. 20A is a sectional view showing the sectional shape of a frame including the center of a first light receiver of the biological information detection device according to the fourth embodiment.

FIG. 20B is a sectional view showing the sectional shape of a frame including the center of a second light receiver of the biological information detection device according to the fourth embodiment.

FIG. 21 is a plan view showing an example of the configuration of a detector according to a first modification of the fourth embodiment.

FIG. 22 is a plan view showing an example of the configuration of a detector according to a second modification of the fourth embodiment.

FIG. 23 is a plan view showing the configuration of a detector of a biological information detection device according to a fifth embodiment of the invention.

FIG. 24 is a sectional view showing the configuration of the detector in the fifth embodiment.

FIG. 25 is a plan view showing an example of the configuration of a detector according to a modification of the fifth embodiment.

FIG. 26 is a plan view showing the configuration of a detector of a biological information detection device according to a sixth embodiment of the invention.

FIG. 27 is a sectional view showing the configuration of a detector of a biological information detection device according to a seventh embodiment of the invention.

### DESCRIPTION OF EXEMPLARY EMBODIMENTS

### First Embodiment

A first embodiment of the invention is explained below with reference to the drawings.

### Configuration of a Biological Information Detection Device

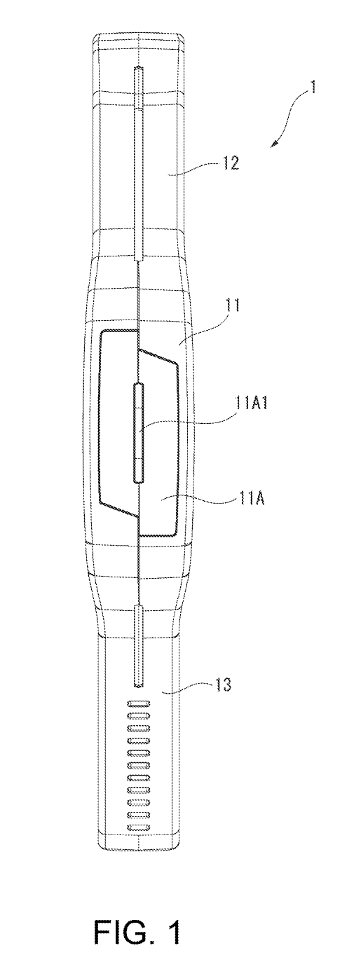

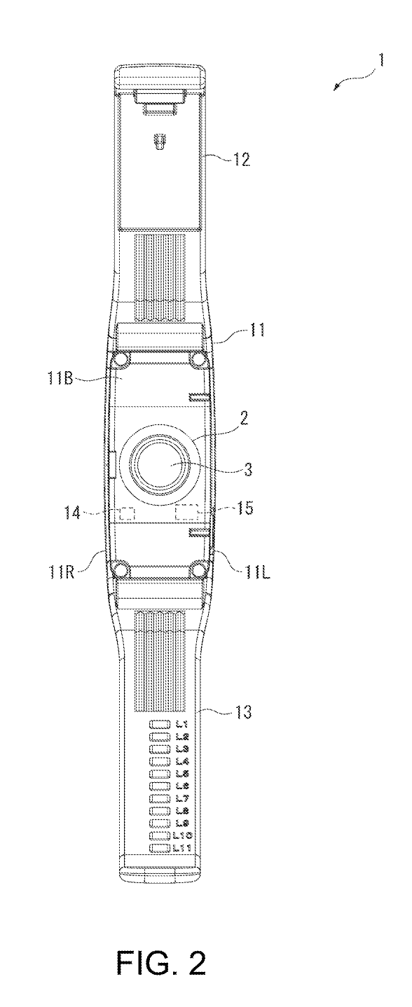

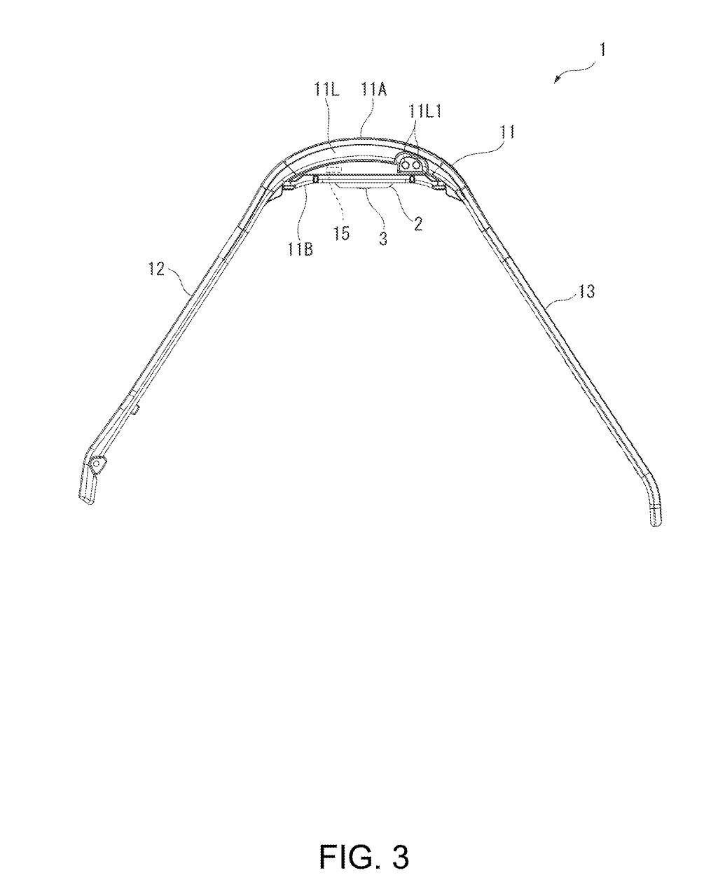

FIG. 1 is a front view showing a biological information detection device 1 according to this embodiment. FIGS. 2 and 3 are respectively a rear view and a side view showing the biological information detection device 1.

The biological information detection device (hereinafter sometimes simply referred to as detection device) 1 according to this embodiment is a wearable device worn on a human body (e.g., a wrist) and used. Although not shown in the figure, the detection device 1 is driven by electric power output from a secondary battery incorporated therein. The detection device 1 has a function of storing biological information of a user in a memory and transmitting the biological information stored in the memory to an external apparatus.

The detection device 1 includes, as shown in FIGS. 1 to 3, a device main body 11 and a pair of bands 12 and 13 respectively integrally configured.

The pair of bands 12 and 13 extends in opposite directions each other from one end and the other end in the longitudinal direction of the device main body 11. When the detection device 1 is worn on the wrist, the bands 12 and 13 are combined by a not-shown buckle provided in the band 12, whereby the detection device 1 is fixed to the wrist.

As shown in FIG. 3, the device main body 11 is formed in a substantially arcuate shape in side view corresponding to a part on the outer side of the wrist on which the detection device 1 is worn (a part on the back side of the hand). The device main body 11 includes, as shown in FIGS. 2 and 3, a front section 11A, a back section 11B located on the opposite side of the front section 11A, and left and right side sections 11L and 11R respectively crossing the front section 11A and the back section 11B. The device main body 11 is formed in a substantially rectangular shape viewed from the front section 11A side.

In the front section 11A, a display 11A1 that displays a state of the detection device 1 is provided. Although not shown in the figure, in the front section 11A, a pattern such as a logo of a manufacturer that manufactures the detection device 1 is provided.

The left and right side sections 11L and 11R are a pair of side sections extending along an extending direction of the bands 12 and 13 respectively extending in opposite directions from the device main body 11.

The left side section 11L (the left side section 11L located on the left side in a drawing view of FIG. 1) includes, as shown in FIG. 3, in a position on the band 13 side, a terminal 11L1 electrically connected to a not-shown cradle.

The back section 11B includes, as shown in FIG. 3, a contactor 2 and a detector 3.

The contactor 2 is a part in contact with the wrist of the user (specifically, the skin of the wrist). The detector 3 is disposed in the center portion of the contactor 2.

The detector 3 has a function of detecting biological information such as a pulse wave of the user. The detector 3 is provided to project from the center portion of the contactor 2. The detector 3 is equivalent to the biological information detection sensor according to the invention.

The device main body 11 includes, as shown in FIG. 2, an acceleration sensor 14 and a controller 15. The acceleration sensor 14 has a function of acquiring a signal concerning noise due to a body motion of the user. The controller 15 is electrically connected to the detector 3 and the acceleration sensor 14. The controller 15 analyzes a detection result by the detector 3 and calculates a pulse rate of the user.

### Configuration of the Detector

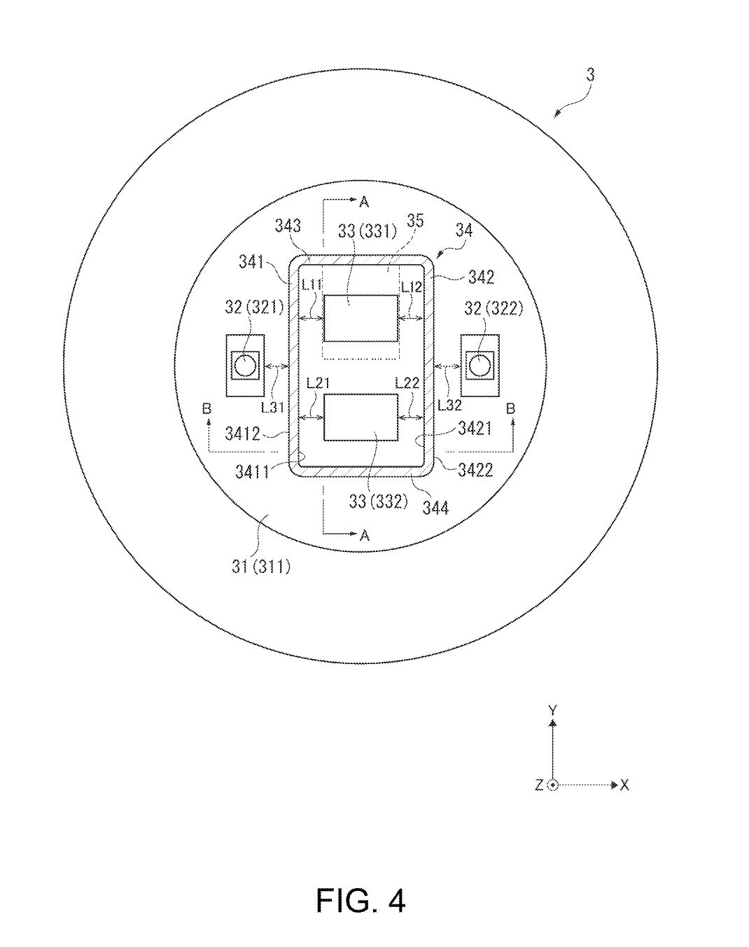

FIG. 4 is a plan view of the detector 3 viewed from the back section 11B side.

The detector 3 includes a substrate 31, light emitters 32, light receivers 33, and a frame (a light blocker) 34.

The substrate 31 is equivalent to the supporting body according to the invention. As shown in FIG. 4, the light emitters 32, the light receivers 33, and the frame 34 are disposed on a surface of the substrate 31 opposed to the detector 3 when the detection device 1 is worn.

Note that, in the following explanation, a Z direction is a direction away from the back section 11B along the normal of the back section 11B. An X direction and a Y direction are directions orthogonal to the Z direction and orthogonal to each other. Among the directions, the X direction is a direction from the right side section 11R to the left side section 11L of the detection device 1. The Y direction is a direction from an attachment position of the band 13 to the device main body 11 of the detection device 1 to an attachment position of the band 12 to the device main body 11. Further, a Z-direction side indicates a downstream side in the Z direction (a Z-direction distal end side). The opposite side of the Z direction indicates an upstream side in the Z direction (a Z-direction proximal end side). The same applies to the other directions.

The light emitters 32 include a first light emitter 321 and a second light emitter 322. The light emitters 32 have a function of emitting lights to the wrist (a wearing part) of the user wearing the detection device 1. In this embodiment, the light emitters 321 and 322 are configured by LEDs (Light Emitting Diodes). The wavelength band of lights emitted from the light emitters 321 and 322 are set to a wavelength band included in a range of, for example, 470 nm or more and 610 nm or less. More specifically, the wavelength band is set to a wavelength band included in a range of 520 nm or more and 570 nm or less that is easily reflected by hemoglobin in a blood vessel of the user. Specifically, the light emitters 321 and 322 emit green lights. Consequently, the lights emitted from the light emitters 32 are reflected by the hemoglobin in the blood vessel of the wrist and received by the light receivers 33.

Note that the lights respectively emitted from the first light emitter 321 and the second light emitter 322 are set in the same waveband. However, a wavelength for maximizing intensity only has to be within a predetermined range (e.g., a range of the same color).

The light receivers 33 include a first light receiver 331 and a second light receiver 332. The light receivers 33 have a function of photodetectors that detect the lights emitted from the light emitters 321 and 322 and reflected by the hemoglobin in the blood vessel. That is, the first light receiver 331 receives the lights emitted from the light emitters 321 and 322 and reflected. Similarly, the second light receiver 332 receives the lights emitted from the light emitters 321 and 322 and reflected. In this embodiment, the light receivers 331 and 332 are configured by PDs (Photodiodes). The lights received by the light receivers 331 and 332 are output to the controller 15 as detection signals.

As shown in FIG. 4, when viewed from the Z-direction side (i.e., the detection part side), the frame 34 is a wall-like convex structure formed in a substantially rectangular cylinder shape, which includes side portions 341, 342, 343, and 344, and extending in the Z direction from the substrate 31. The frame 34 is disposed between the light emitters 321 and 322 and the light receivers 331 and 332. The frame 34 has a function of blocking lights emitted from the light emitters 321 and 322 and made directly incident on the light receivers 331 and 332. That is, the frame 34 is equivalent to the light blocker according to the invention.

Disposition configuration of the light emitters, the light receivers, and the frame

As shown in FIG. 4, the light emitters 321 and 322, the light receivers 331 and 332, and the frame 34 are mounted (disposed) on a mounting surface 311 of the substrate 31.

Specifically, the first light receiver 331 and the second light receiver 332 are disposed side by side along the Y direction in a center region on the mounting surface 311 of the substrate 31. The frame 34 is disposed on the mounting surface 311 of the substrate 31 to surround the light receivers 331 and 332 in plan view (when viewed from the Z-direction side).

The first light emitter 321 and the second light emitter 322 are disposed along the X direction on the outer side of the frame 34 on the mounting surface 311. Specifically, the first light emitter 321 is disposed on the opposite direction side of the X direction with respect to the side portion 341 of the frame 34 on the mounting surface 311. The second light emitter 322 is disposed on the X-direction side with respect to the side portion 342 on the mounting surface 311. The light emitters 321 and 322 are disposed in an intermediate position of the first light receiver 331 and the second light receiver 332, that is, a position where a linear distance from the first light receiver 331 to the light emitters 321 and 322 and a linear distance from the second light receiver 332 to the light emitters 321 and 322 are the same.

A distance L11 from the first light receiver 331 to an inner surface 3411 of the side portion 341 and a distance L12 from the first light receiver 331 to an inner surface 3421 of the side portion 342 are set to the same distance. A distance L21 from the second light receiver 332 to the inner surface 3411 and a distance L22 from the second light receiver 332 to the inner surface 3421 are set to the same distance.

Further, a distance L31 from the first light emitter 321 to an outer surface 3412 of the side portion 341 and a distance L32 from the second light emitter 322 to an outer surface 3422 of the side portion 342 are set to the same distance.

That is, when viewed from the Z-direction side, the distance between the first and second light emitters 321 and 322 and the first light receiver 331 is the same as the distance between the first and second light emitters 321 and 322 and the second light receiver 332.

In other words, in plan view (when viewed from the Z-direction side), a straight line connecting the center of the first light receiver 331 and the center of the second light receiver 332 and a straight line connecting the center of the first light emitter 321 and the center of the second light emitter 322 are disposed to be orthogonal to each other.

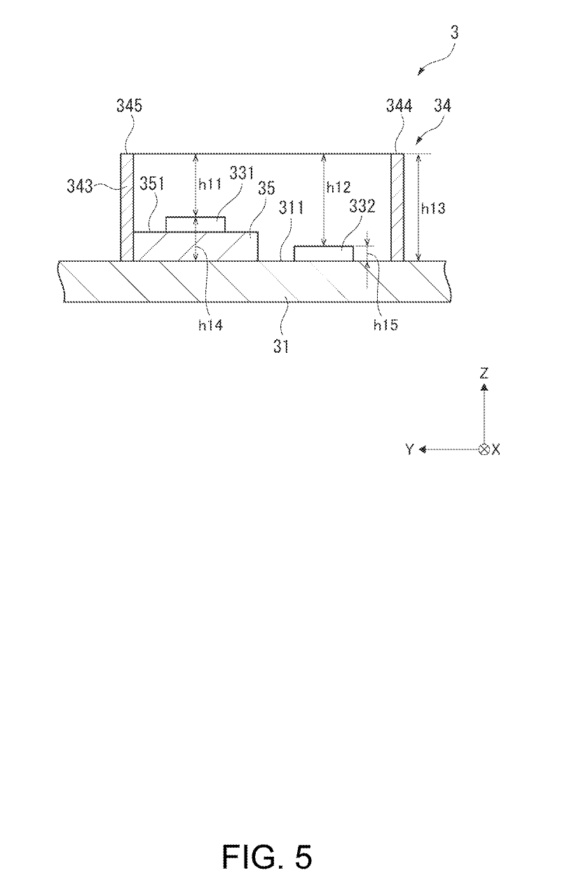

FIG. 5 is a sectional view of an A-A section in FIG. 4 in the detector 3 viewed from the right side section 11R side.

Distances to the first light receiver 331 and the second light receiver 332 from the mounting surface 311 are different from each other as shown in FIG. 5.

Specifically, a rectangular plate-like pedestal 35 is disposed on the mounting surface 311 of the substrate 31. The first light receiver 331 is disposed on an upper surface 351 of the pedestal 35. Therefore, a distance h11 from the first light receiver 331 to an upper end face 345 of the frame 34 is shorter than a distance h12 from the second light receiver 332 to the upper end face 345. In other words, the first light receiver 331 and the second light receiver 332 are respectively disposed in positions at different distances from the mounting surface 311 of the substrate 31. A distance h13 from the substrate 31 to the upper end face 345 of the frame 34 is longer than distances h14 and h15 from the substrate 31 to the light receivers 331 and 332.

In this way, the first light receiver 331 and the second light receiver 332 are respectively disposed in positions on the mounting surface 311 where lengths or routes of an optical path of light emitted from the first light emitter 321, reflected in the wearing part (the detection part), and received by the light receivers 331 and 332 are different from each other and lengths or routes of an optical path of light emitted from the second light emitter 322, reflected in the wearing part (the detection part), and received by the light receivers 331 and 332 are different from each other.

### Noise Reduction Processing

Body motion noise reduction processing performed in the controller 15 is explained. Specifically, a spectral subtraction method performed on the basis of a detection signal (hereinafter sometimes referred to as second detection signal) received by the second light receiver 332 and adaptive filter processing performed on the basis of a signal from the acceleration sensor 14 are explained. Note that, in the following explanation, a detection signal received by the first light receiver 331 is sometimes referred to as first detection signal.

### Spectral Subtraction Method

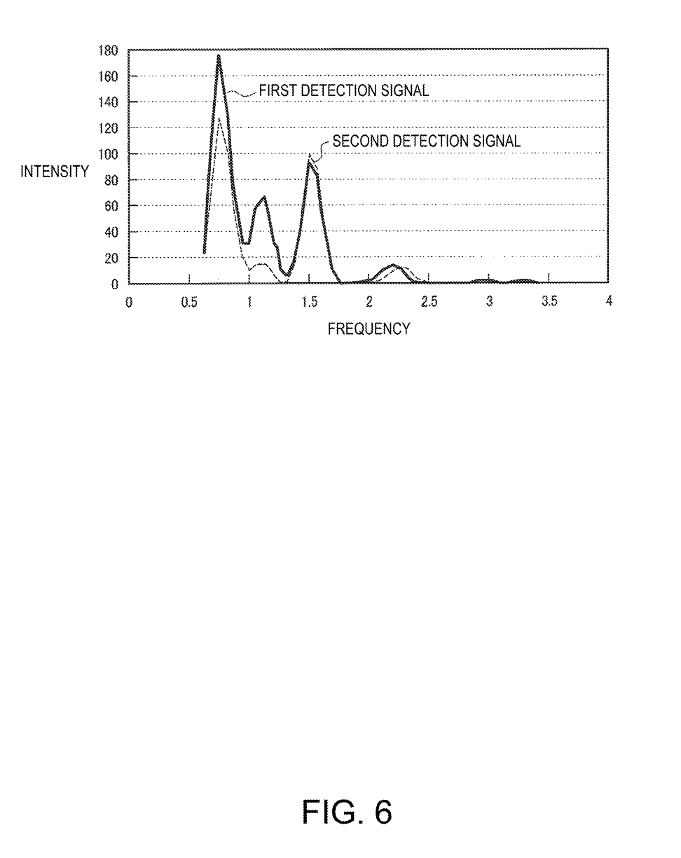

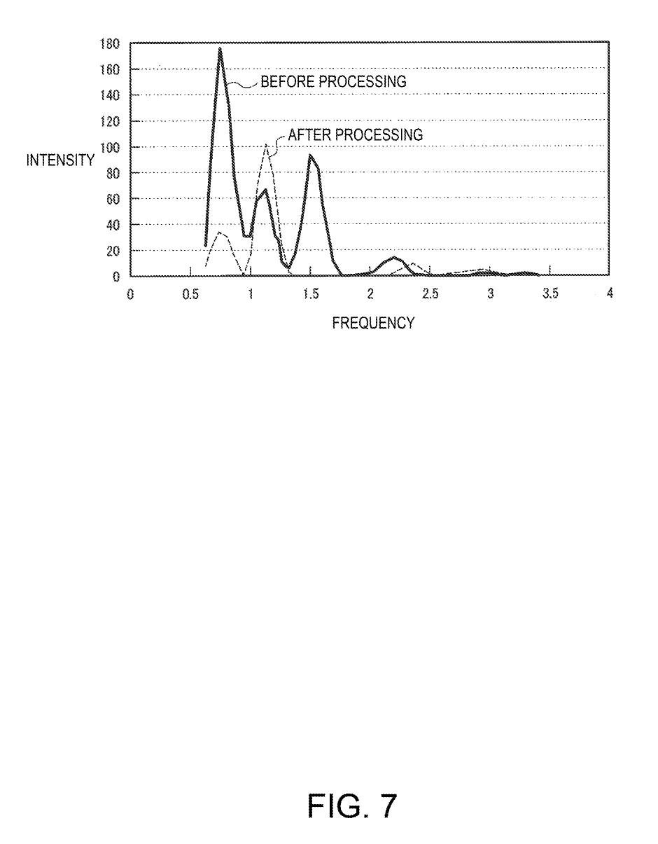

FIGS. 6 and 7 are diagrams for explaining noise reduction processing for the first detection signal based on the second detection signal performed using the spectral subtraction method. In the spectral subtraction method, frequency conversion processing is performed on the first and second detection signals to respectively calculate spectra. Processing for estimating a noise spectrum from the spectrum of the second detection signal and subtracting the estimated noise spectrum from the spectrum of the first detection signal is performed.

In FIG. 6, a spectrum of the first detection signal and a spectrum of the second detection signal actually calculated by the spectral subtraction method are shown. As explained above, when the biological information detection device 1 according to this embodiment is used, the spectrum of the second detection signal changes to a spectrum mainly corresponding to a noise component. That is, it can be estimated that a frequency at a large peak in the spectrum of the second detection signal is a frequency corresponding to body motion noise. Actually, only peaks may be subtracted in the spectrum of the second detection signal. However, the invention is not limited to this. For example, processing for subtracting the entire spectrum of the second detection signal from the entire spectrum of the first detection signal may be performed.

In the subtraction, to offset noise, for example, one of the first detection signal and the second detection signal is multiplied with a coefficient. The coefficient is calculated from, for example, signal intensity at a predetermined frequency. Alternatively, it is also possible that noise and a signal are separated by a method such as clustering and the coefficient is calculated to set noise of the first detection signal and noise of the second detection signal to the same intensity.

In FIG. 7, an example of the first detection signal before and after the body motion noise reduction processing by the spectral subtraction method is shown. Body motion noise appearing at 0.7 to 0.8 Hz (42 to 48 in terms of a pulse rate) and 1.5 Hz (a pulse rate of 90) is reduced by the body motion noise reduction processing as shown in FIG. 7. It is possible to suppress the possibility that the body motion noise is erroneously determined as pulse signals. On the other hand, it is possible to maintain a signal level without reducing the signal level concerning spectra corresponding to pulse signals appearing before and after 1.1 Hz (a pulse rate of 66).

The spectral subtraction method is realized by frequency conversion processing such as FFT (Fast Fourier Transform) and subtraction processing in a spectrum. Therefore, there is an advantage that an algorithm is simple and computational complexity is small. Since the spectral subtraction method does not include learning elements like those of adaptive filter processing explained below, the spectral subtraction method has a characteristic that instantaneous responsiveness is high.

Adaptive Filter Processing

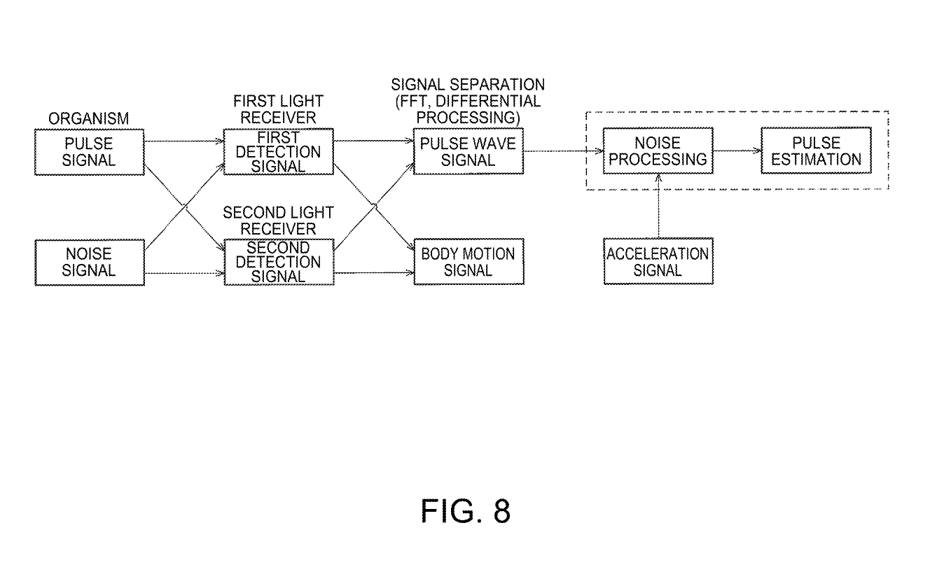

Body motion noise reduction processing (second body motion noise reduction processing) based on a detection signal from the acceleration sensor 14 performed using the adaptive filter processing is explained. A specific example of noise reduction processing performed using an adaptive filter is shown in FIG. 8. Specifically, since the detection signal of the acceleration sensor 14 corresponds to body motion noise, processing for subtracting a noise component specified from the detection signal from the first detection signal is performed. A rough idea of the noise reduction processing is the same as the spectral subtraction method.

However, even if both of body motion noise in a pulse wave detection signal and the detection signal (a body motion detection signal) from the acceleration sensor 14 are signals due to the same body motion, signal levels of the body motion noise and the detection signal are not always the same. Therefore, filter processing, a filter coefficient of which is adaptively determined, is performed on the body motion detection signal to calculate an estimated body motion noise component and calculate a difference between the pulse wave detection signal and the estimated body motion noise component. Since the filter coefficient is adaptively determined (by performing learning), it is possible to improve the accuracy of the noise reduction processing. However, it is necessary to take into account a processing load in the determination of the filter coefficient and a delay of an output. Note that, since the adaptive filter processing is a widely known method, detailed explanation of the adaptive filter processing is omitted.

In this embodiment, the biological information detection device 1 includes the acceleration sensor 14 as shown in FIG. 2. The controller 15 performs, on the basis of a detection signal from the acceleration sensor 14, the second body motion noise reduction processing for reducing the body motion noise of the first detection signal.

That is, this embodiment is based on the premise that the body motion noise reduction processing is performed using the second detection signal from the second light receiver 332. However, the body motion noise reduction processing may be performed concurrently using the acceleration sensor 14. This makes it possible to more accurately reduce the body motion noise compared with when the body motion noise reduction processing is performed using the second detection signal only. For example, in FIG. 6, noise at 0.7 to 0.8 Hz or 2.3 to 2.4 Hz is not fully reduced. However, it is also possible to reduce the noise concurrently using processing performed by using a detection signal from a motion sensor.

The controller 15 may perform the body motion noise reduction processing for the first detection signal on the basis of the second detection signal and perform the second body motion noise reduction processing on a signal after the body motion noise reduction processing on the basis of the detection signal from the acceleration sensor 14.

Consequently, it is possible to perform a plurality of kinds of body motion noise reduction processing in predetermined order. In this embodiment, first, the body motion noise reduction processing is performed using the second detection signal. Thereafter, the second body motion noise reduction processing is performed. A flow of signals in this case is shown in FIG. 7.

A pulse signal and a noise signal can be detected from an organism. As shown in FIG. 7, both of the pulse signal and the noise signal are included in detection signals from the first light receiver 331 and the second light receiver 332. However, in this embodiment, a ratio of the pulse signal and the noise signal are different for each of the light receivers 331 and 332. The first detection signal includes the pulse signal at a relatively high ratio. In the second detection signal, a ratio of the pulse signal is low compared with the first detection signal (a ratio of the body motion noise is high). The pulse signal and the body motion signal (the body motion noise) are separated using the two detection signals. This processing is realized by the spectral subtraction method. The second body motion noise reduction processing is performed on the separated pulse signal (the first detection signal after the body motion noise reduction processing) using the detection signal (in FIG. 7, an acceleration signal) of the acceleration sensor 14. A pulse rate and the like are estimated from a result of the second body motion noise reduction processing.

### Effects of the First Embodiment

The biological information detection device 1 according to this embodiment explained above has effects explained below.

According to this embodiment, the frame (the light blocker) 34 is disposed between the light emitters 321 and 322 and the light receivers 331 and 332. Therefore, it is possible to suppress lights emitted from the light emitters 321 and 322 from being made directly incident on and received by the light receivers 331 and 332. The controller 15 can analyze a pulse wave serving as biological information on the basis of light reception results by the light receivers 331 and 332 of the lights emitted from the light emitters 321 and 322 and reflected in the detection part. Therefore, it is possible to accurately detect biological information (a pulse wave) compared with a biological information measuring device in which only one light receiving element is provided like the biological information detection device described in Patent Literature 1.

Since the light receivers 331 and 332 are surrounded by the frame 34, it is possible to further reduce the possibility that the lights emitted from the light emitters 321 and 322 are made directly incident on and received by the light receivers 331 and 332. It is possible to suppress external light deriving from a portion other than the light emitters 321 and 322 from being made incident on the light receivers 331 and 332. Therefore, since the light receivers 331 and 332 can surely receive the lights made incident via the detection part, it is possible to more accurately detect biological information.

Light reception results of the lights made incident via the detection part can be differentiated from each other by the first light receiver 331 and the second light receiver 332. In other words, the first light receiver 331 and the second light receiver 332 can receive the lights having the different kinds of information. Consequently, the controller 15 can analyze the biological information on the basis of the light reception results received from the first light receiver 331 and the second light receiver 332. Therefore, it is possible to further improve the detection and analysis accuracy of biological information.

The first light receiver 331 and the second light receiver 332 are respectively disposed in the positions at the different distances from the mounting surface 311 of the substrate 31. Therefore, it is possible to surely differentiate the lengths of the optical paths or the routes of the lights emitted from the first light emitter 321 and the second light emitter 322 and made incident on the first light receiver 331 and the second light receiver 332 via the detection part. In other words, the first light receiver 331 and the second light receiver 332 can receive the lights having the different kinds of information. Therefore, since it is possible to surely differentiate the light reception results by the first light receiver 331 and the second light receiver 332, it is possible to surely improve the detection and analysis accuracy of biological information.

The distance h13 from the substrate 31 to the upper end face 345 of the frame 34 is longer than the distances h14 and h15 from the substrate 31 to the light receiving surfaces of the light receivers 331 and 332. Therefore, it is possible to further reduce the possibility that the lights emitted from the light emitters 321 and 322 are made directly incident on and received by the light receivers 331 and 332. It is possible to further suppress external light deriving from a portion other than the light emitters 321 and 322 from being made incident on the light receivers 331 and 332. Therefore, since the light receivers 331 and 332 can surely receive the lights made incident via the detection part, it is possible to more accurately detect biological information.

### Modification of the First Embodiment

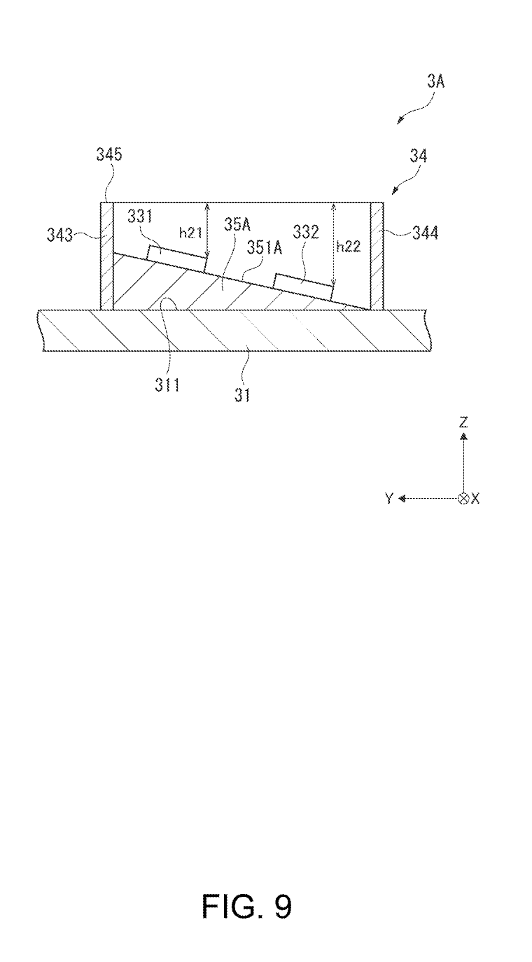

FIG. 9 is a sectional view of the A-A section in FIG. 4 in a detector 3A in a modification of this embodiment viewed from the right side section 11R side.

In the first embodiment, the first light receiver 331 is disposed on the upper surface 351 of the rectangular plate-like pedestal 35. However, the invention is not limited to this. For example, the biological information detection device 1 may adopt a detector 3A shown in FIG. 9 instead of the detector 3.

Specifically, the detector 3A includes a triangular prism-like pedestal 35A disposed on the mounting surface 311 of the substrate 31. The pedestal 35A has a slope 351A. The slope 351A inclines in the opposite direction of the Z direction from the Y direction side toward the opposite side of the Y direction.

On the slope 351A, as in the first embodiment, the first light receiver 331 and the second light receiver 332 are disposed. In other words, the first light receiver 331 and the second light receiver 332 are respectively disposed in positions at different distances from the mounting surface 311 of the substrate 31.

As explained above, the first light receiver 331 is disposed on the slope 351A of the pedestal 35A. Therefore, a distance h21 from the first light receiver 331 to the upper end face 345 of the frame 34 is shorter than a distance h22 from the second light receiver 332 to the upper end face 345 as shown in FIG. 9. In other words, the first light receiver 331 and the second light receiver 332 are respectively disposed in positions at different distances from the mounting surface 311 of the substrate 31.

In this way, the first light receiver 331 and the second light receiver 332 are respectively disposed in positions on the mounting surface 311 where lengths of optical paths of the light emitted from the first light emitter 321, reflected in the wearing part (the detection part), and received by the light receivers 331 and 332 are different from each other and lengths of optical paths of the light emitted from the second light emitter 322, reflected in the wearing part (the detection part), and received by the light receivers 331 and 332 are different from each other.

Therefore, in this modification, it is possible to achieve effects same as the effects of the first embodiment.

### Second Embodiment

A second embodiment of the invention is explained.

A biological information detection device according to this embodiment includes a configuration same as the configuration of the biological information detection device 1. However, the configuration of the detector 3 is different. That is, in this embodiment, the detector 3 includes a frame having a shape different from the shape of the frame 34. In the first embodiment, the detector 3 includes the pedestal 35 on the mounting surface 311 of the substrate 31. However, in this embodiment, the detector 3 does not include the pedestal 35. The biological information detection device according to this embodiment and the biological information detection device 1 are different in these points. Note that, in the following explanation, portions the same as or substantially the same as the portions already explained above are denoted by the same reference numerals and signs and explanation of the portions is omitted.

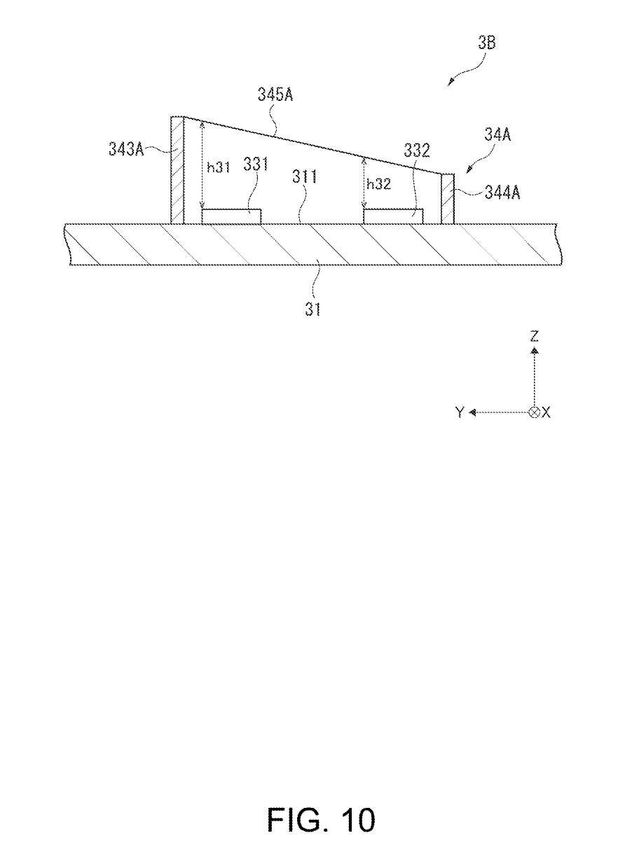

FIG. 10 is a sectional view of the A-A section (a cross section in an array direction of the light receivers 331 and 332) in FIG. 4 in a detector 3B in this embodiment viewed from the right side section 11R side.

The biological information detection device according to this embodiment includes components and functions same as those of the biological information detection device 1 except that the biological information detection device includes the detector 3B instead of the detector 3. The detector 3B includes a frame 34A instead of the frame 34. The first light receiver 331 is directly disposed on the mounting surface 311 of the substrate 31.

The frame 34A includes, as shown in FIG. 10, a side portion 343A and a side portion 344A having different heights. The height in the Z direction of the side portion 343A is larger than the height in the Z direction of the side portion 344A. That is, an upper end face 345A of the frame 34A inclines in the opposite direction of the Z direction from the Y-direction side toward the opposite side of the Y direction. The sectional shape of the side portion 343A on the first light receiver 331 side in the frame 34A and the sectional shape of the side portion 344A on the second light receiver 332 side are different. In other words, the area of a cross section extending along a YZ plane of the side portion 343A in the frame 34A is larger than the area of a cross section extending along the YZ plane of the side portion 344A in the frame 34A.

As explained above, the upper end face 345A of the frame 34A inclines. Therefore, a distance h31 from the first light receiver 331 to the upper end face 345A of the frame 34A is longer than a distance h32 from the second light receiver 332 to the upper end face 345A.

In this way, the distance h31 and the distance h32 are different. Therefore, as in the case in which the dimensions of the first light receiver 331 and the second light receiver 332 from the mounting surface 311 of the substrate 31 in the first embodiment are different, lengths of optical paths of the light emitted from the first light emitter 321, reflected in the wearing part (the detection part), and received by the light receivers 331 and 332 are different from each other and lengths of optical paths of the light emitted from the second light emitter 322, reflected in the wearing part (the detection part), and received by the light receivers 331 and 332 are different from each other.

### Effects of the Second Embodiment

The biological information detection device according to this embodiment explained above has effects explained below besides effects same as the effects of the biological information detection device 1 in the first embodiment.

According to this embodiment, the sectional shape of the side portion 343A on the first light receiver 331 side in the frame 34A and the sectional shape of the side portion 344A on the second light receiver 332 side are different. Therefore, it is possible to differentiate lengths of optical paths or routes of light including different kinds of information emitted from the light emitters 321 and 322 and made incident on the first light receiver 331 and the second light receiver 332 via the detection part. In other words, the first light receiver 331 and the second light receiver 332 can receive the lights having the different kinds of information. Therefore, since it is possible to differentiate light reception results by the first light receiver 331 and the second light receiver 332, it is possible to surely improve the detection and analysis accuracy of biological information.

### Modifications of the Second Embodiment

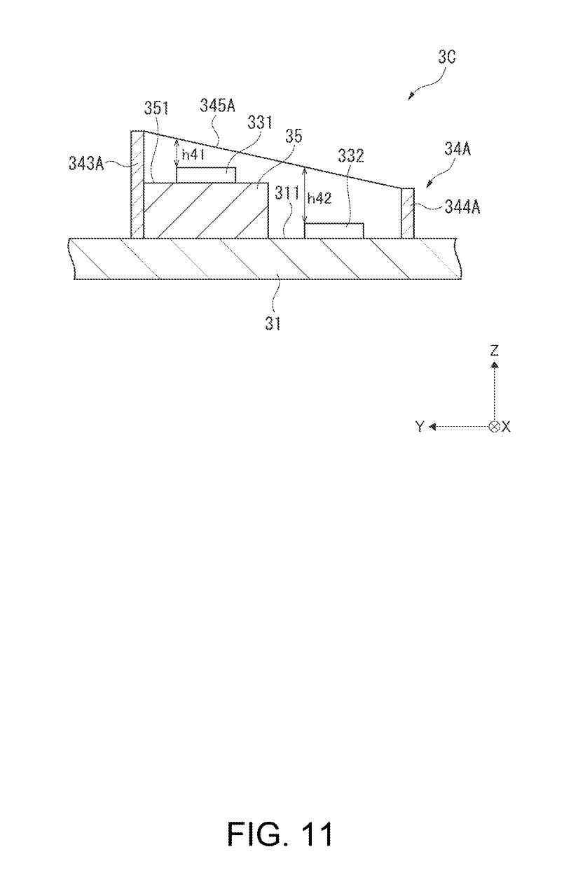

FIG. 11 is a sectional view of the A-A section in FIG. 4 in a detector 3C in a first modification of this embodiment viewed from the right side section 11R side.

In the second embodiment, the first light receiver 331 is directly disposed on the mounting surface 311 of the substrate 31. However, the invention is not limited to this. For example, the biological information detection device may adopt the detector 3C shown in FIG. 11 instead of the detector 3B.

Specifically, in the detector 3C, the rectangular plate-like pedestal 35 disposed on the mounting surface 311 of the substrate 31 may be provided. The first light receiver 331 may be disposed on the upper surface 351 of the pedestal 35.

As explained above, the first light receiver 331 is disposed on the upper surface 351 of the pedestal 35. Therefore, a distance h41 from the first light receiver 331 to the upper end face 345A of the frame 34A is shorter than a distance h42 from the second light receiver 332 to the upper end face 345A. In other words, the first light receiver 331 and the second light receiver 332 are respectively disposed in positions at different distances from the mounting surface 311 of the substrate 31.

In this way, in the first light receiver 331 and the second light receiver 332, lengths of optical paths of the light emitted from the first light emitter 321, reflected in the wearing part (the detection part), and received by the light receivers 331 and 332 are different from each other and lengths of optical paths or routes of the light including the different kinds of information emitted from the second light emitter 332, reflected in the wearing part (the detection part), and made incident on the light receivers 331 and 332 are different from each other. In other words, the first light receiver 331 and the second light receiver 332 can receive the lights having the different kinds of information. Therefore, in the first modification, it is possible to achieve effects same as the effects of the second embodiment.

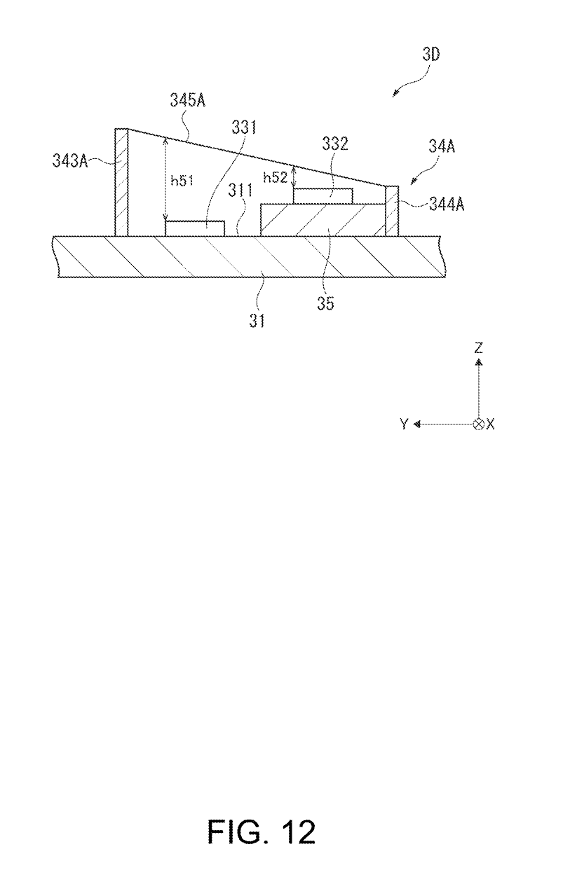

FIG. 12 is a sectional view of the A-A section in FIG. 4 in a detector 3D in a second modification of this embodiment viewed from the right side section 11R side.

In the first modification, the first light receiver 331 is disposed on the upper surface 351 of the pedestal 35. However, the invention is not limited to this. For example, the biological information detection device may adopt the detector 3D shown in FIG. 12 instead of the detector 3C.

Specifically, in the detector 3D, the rectangular plate-like pedestal 35 disposed on the mounting surface 311 of the substrate 31 may be provided. The second light receiver 332 may be disposed on the upper surface 351 of the pedestal 35.

Consequently, a distance h51 from the first light receiver 331 to the upper end face 345A of the frame 34A is shorter than a distance h52 from the second light receiver 332 to the upper end face 345A. A difference between the distance h51 and the distance h52 can be set larger than a difference between the distance h41 and the distance h42 in the first modification. Therefore, compared with the first modification, it is possible to further differentiate the lengths of the optical paths or the routes of the light respectively received by the first light receiver 331 and the second light receiver 332.

Therefore, in the second modification, it is possible to achieve effects same as the effects of the second embodiment.

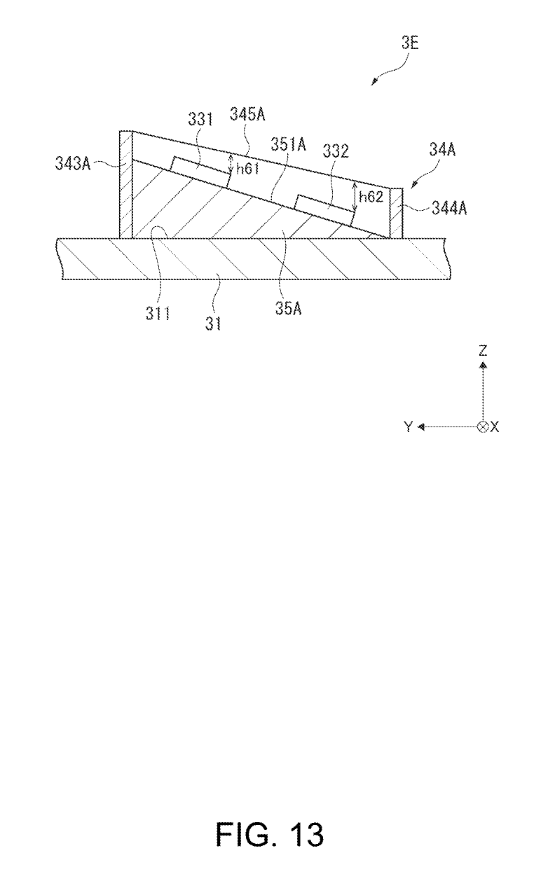

FIG. 13 is a sectional view of the A-A section in FIG. 4 in a detector 3E in a third modification of this embodiment viewed from the right side section 11R side.

In the second embodiment, the first light receiver 331 is directly disposed on the mounting surface 311 of the substrate 31. However, the invention is not limited to this. For example, the biological information detection device may adopt the detector 3E shown in FIG. 13 instead of the detector 3B.

Specifically, in the detector 3E, the triangular prism-like pedestal 35A is provided on the mounting surface 311 of the substrate 31. The first light receiver 331 and the second light receiver 332 are disposed on the slope 351A of the pedestal 35A. The pedestal 35A has the slope 351A. The slope 351A inclines in the opposite direction of the Z direction from the Y-direction side toward the opposite side of the Y direction. That is, the first light receiver 331 and the second light receiver 332 are respectively disposed in positions at different distances from the mounting surface 311 of the substrate 31.

As explained above, the first light receiver 331 is disposed on the slope 351A of the pedestal 35A. Therefore, a distance h61 from the first light receiver 331 to the upper end face 345A of the frame 34A is shorter than a distance h62 from the second light receiver 332 to the upper end face 345A. In other words, the first light receiver 331 and the second light receiver 332 are respectively disposed in positions at different distances from the mounting surface 311 of the substrate 31.

In this way, in the first light receiver 331 and the second light receiver 332, lengths of optical paths or routes of the light including the different kinds of information emitted from the first light emitter 321, reflected in the wearing part (the detection part), and made incident on the light receivers 331 and 332 are different from each other and lengths of optical paths of the light emitted from the second light emitter 322, reflected in the wearing part (the detection part), and made incident on the light receivers 331 and 332 are different from each other. In other words, the first light receiver 331 and the second light receiver 332 can receive the lights having the different kinds of information. Therefore, in the third modification, it is possible to achieve effects same as the effects of the second embodiment.

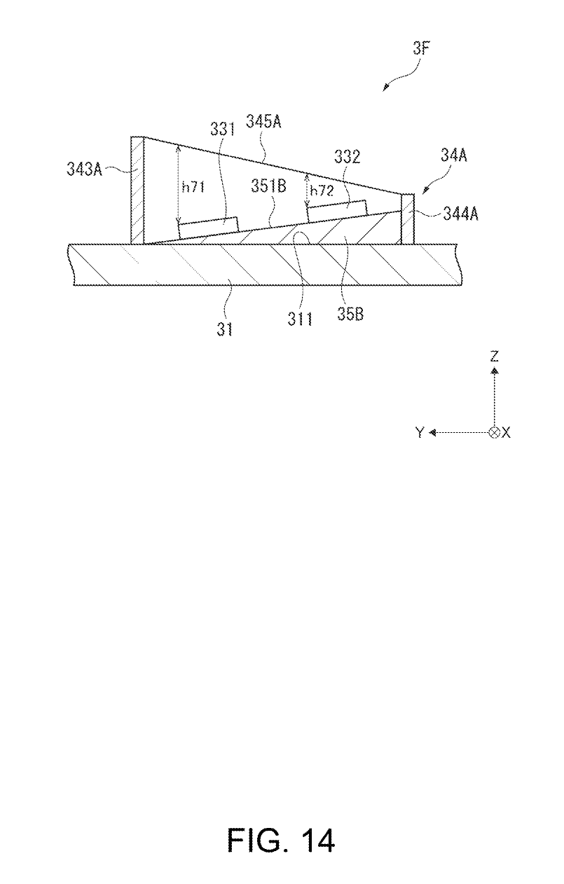

FIG. 14 is a sectional view of the A-A section in FIG. 4 in a detector 3F in a fourth modification of this embodiment viewed from the right side section 11R side.

In the third modification, the first light receiver 331 and the second light receiver 332 are disposed on the slope 351A of the pedestal 35A. However, the invention is not limited to this. For example, the biological information detection device may adopt the detector 3F shown in FIG. 14 instead of the detector 3E.

Specifically, in the detector 3F, a pedestal 35B including a slope 351B inclining in the opposite direction of the slope 351A of the pedestal 35A is provided on the mounting surface 311 of the substrate 31. The light receivers 331 and 332 are disposed on the slope 351B of the pedestal 35B.

In this way, a distance h71 from the first light receiver 331 to the upper end face 345A of the frame 34A is shorter than a distance h72 from the second light receiver 332 to the upper end face 345A. A difference between the distance h71 and the distance h72 can be set larger than a difference between the distance h61 and the distance h62 in the third modification. Therefore, compared with the third modification, it is possible to further differentiate the lengths of the optical paths or the routes of the lights respectively received by the first light receiver 331 and the second light receiver 332.

Therefore, in the fourth modification, it is possible to achieve effects same as the effects of the second embodiment.

### Third Embodiment

A third embodiment of the invention is explained below.

A biological information detection device according to this embodiment includes a configuration same as the configuration of the biological information detection device 1. However, the configuration of the detector 3 is different. That is, in this embodiment, the detector 3 includes a pedestal having a shape different from the shape of the pedestal 35. In the first embodiment, the first light receiver 331 is disposed on the upper surface 351 of the pedestal 35. However, in this embodiment, the first light receiver 331 is directly disposed on the mounting surface 311 of the substrate 31. The biological information detection device according to this embodiment and the biological information detection device 1 are different in these points. Note that, in the following explanation, portions the same as or substantially the same as the portions already explained above are denoted by the same reference numerals and signs and explanation of the portions is omitted.

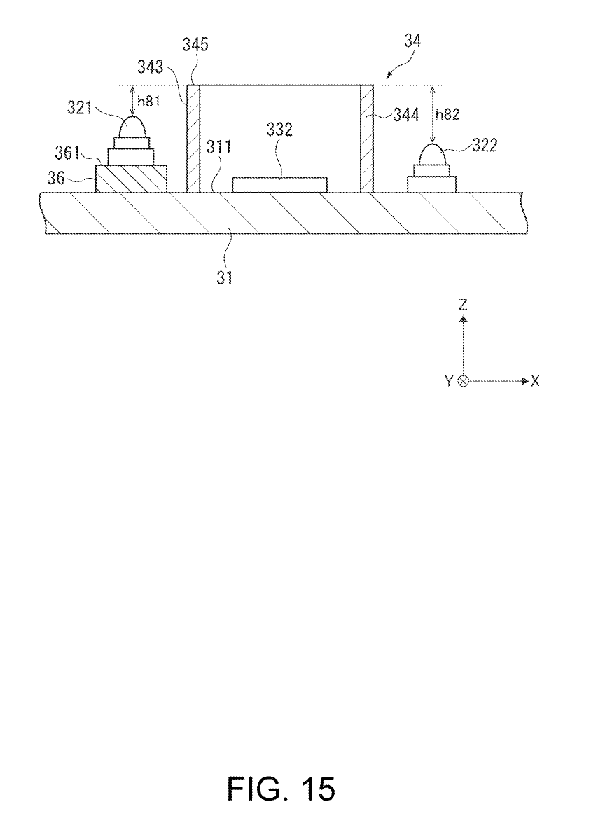

FIG. 15 is a sectional view of a B-B section in FIG. 4 in a detector 3G in this embodiment viewed from the band 13 side.

The biological information detection device according to this embodiment includes components and functions same as those of the biological information detection device 1 except that the biological information detection device includes the detector 3G instead of the detector 3. The detector 3G includes a pedestal 36 instead of the pedestal 35 as shown in FIG. 15. The pedestal 36 is configured in a rectangular plate shape and is disposed in a position on the opposite side of the Z direction in a position where the first light emitter 321 is disposed on the mounting surface 311 of the substrate 31. That is, the first light emitter 321 is disposed on an upper surface 361 of the pedestal 36.

In this way, the first light emitter 321 is disposed on the upper surface 361 of the pedestal 36. Therefore, a distance h81 from the first light emitter 321 to a straight line extending in a direction along the upper end face 345 of the frame 34, that is, a direction along the X direction is shorter than a distance h82 from the second light emitter 322 to a straight line extending in the direction along the upper end face 345. In other words, the first light emitter 321 and the second light emitter 322 are respectively disposed in positions at different distances from the mounting surface 311 of the substrate 31.

That is, the first light emitter 321 and the second light emitter 322 are respectively disposed in the positions at the different distances from the mounting surface 311 of the substrate 31. Therefore, as in the case in which the dimensions of the first light receiver 331 and the second light receiver 332 from the mounting surface 311 of the substrate 31 are different in the first embodiment, lengths of optical paths or routes of the light emitted from the first light emitter 321, reflected in the wearing part (the detection part), and made incident on the light receivers 331 and 332 are different from each other and lengths of optical paths or routes of the light emitted from the second light emitter 322, reflected in the wearing part (the detection part), and made incident on the light receivers 331 and 332 are different from each other.

Note that, in FIG. 15, the upper surface of the first light emitter 321 and the upper surface of the second light emitter 322 further project to the Z-direction side than the upper surface of the first light receiver 331 or the upper surface of the second light receiver 332. That is, the distance between the substrate 31 and the upper surfaces of the light emitters 32 is depicted as being longer than the distance between the substrate 31 and the upper surfaces of the light receivers 33. However, the invention is not limited to this. For example, the upper surface of the first light receiver 331 and the upper surface of the second light receiver 332 may be configured to further project in the Z direction than the upper surface of the first light emitter 321 or the upper surface of the second light emitter 322. That is, the distance between the substrate 31 and the light receiving surfaces of the light receivers 33 may be set longer than the distance between the substrate 31 and the upper surfaces of the light emitters 32. However, the upper end face 345 of the frame 34 is configured to further project in the Z-direction side than the upper surfaces of the light emitters 32 and the upper surfaces of the light receivers 33. With such a configuration, it is possible to differentiate the lengths of the optical paths or the routes of the light emitted from the first light emitter 321 and the light emitted from the second light emitter 322 while applying an appropriate pressing force to the light receiving surfaces. It is possible to improve the detection and analysis accuracy of biological information.

### Effects of the Third Embodiment

The biological information detection device according to this embodiment explained above has effects explained below besides effects same as the effects of the biological information detection device 1 in the embodiments.

According to this embodiment, the first light emitter 321 and the second light emitter 322 are respectively disposed in the positions explained above. Therefore, as explained above, it is possible to surely differentiate the lengths of the optical paths or the routes of the light emitted from the first light emitter 321 and made incident on the first light receiver 331 and the second light receiver 332 via the detection part. In other words, the first light receiver 331 and the second light receiver 332 can receive the lights having the different kinds of information. Therefore, since it is possible to surely differentiate light reception results of the first light receiver 331 and the second light receiver 332, it is possible to surely improve the detection and analysis accuracy of biological information.

### Modifications of the Third Embodiment

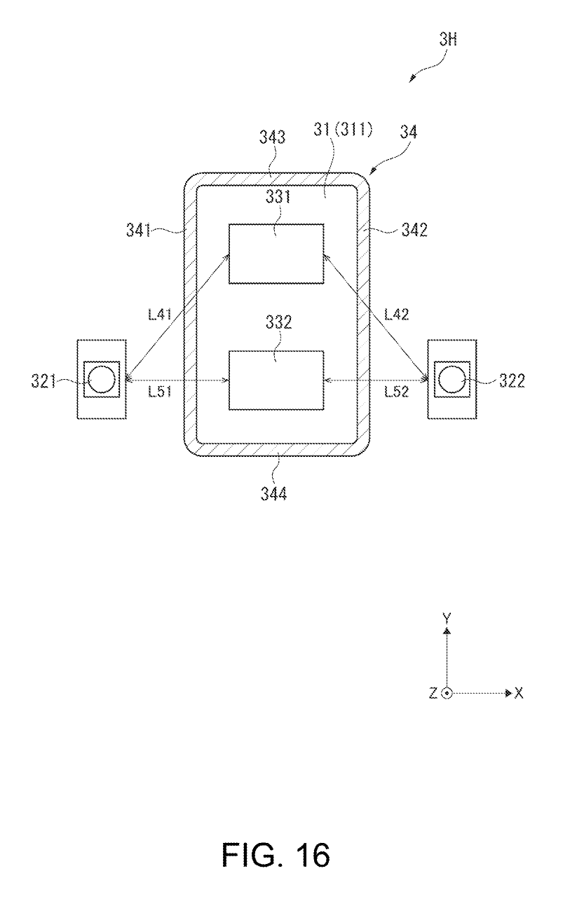

FIG. 16 is a plan view of a detector 3H in a first modification of this embodiment viewed from the back section 11B side.

In the third embodiment, the light emitters 321 and 322 are disposed in the intermediate position of the first light receiver 331 and the second light receiver 332, that is, the position where the linear distance from the first light receiver 331 to the light emitters 321 and 322 and the linear distance from the second light receiver 332 to the light emitters 321 and 322 are the same. However, the invention is not limited to this. For example, the biological information detection device may adopt the detector 3H shown in FIG. 16 instead of the detector 3G.

Specifically, in the detector 3H, the first light emitter 321 and the second light emitter 322 are disposed on the mounting surface 311 of the substrate 31 on a straight line extending along the X direction on which the second light receiver 332 is disposed. In other words, the second light receiver 332 is disposed on a straight line connecting the center of the first light emitter 321 and the center of the second light emitter 322 and the first light receiver 331 is disposed in the vertical direction of the straight line. Therefore, a distance L41 from the first light receiver 331 to the first light emitter 321 and a distance L51 from the second light receiver 332 to the first light emitter 321 are different. A distance L42 from the first light receiver 331 to the second light emitter 322 and a distance L52 from the second light receiver 332 to the second light emitter 322 are different.

That is, since the distances L41 and L42 and the distances L51 and L52 are different, as explained above, it is possible to surely differentiate the lengths of the optical paths or the routes of the light emitted from the first light emitter 321 and made incident on the first light receiver 331 and the second light receiver 332 via the detection part. Therefore, in the first modification, it is possible to achieve effects same as the effects of the third embodiment.

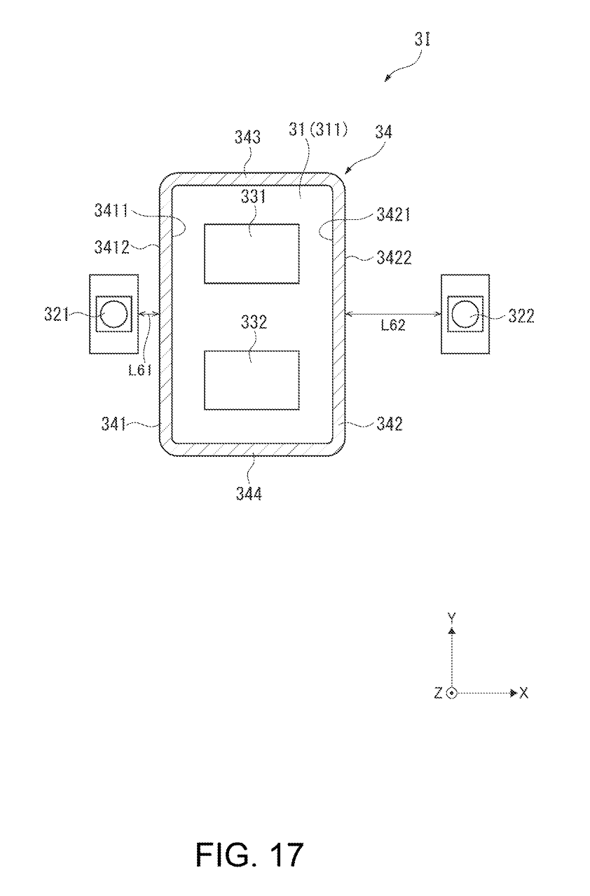

FIG. 17 is a plan view of a detector 3I in a second modification of this embodiment viewed from the back section 11B side.

In the third embodiment, the first light emitter 321 is disposed on the upper surface 361 of the pedestal 36. However, the invention is not limited to this. For example, the biological information detection device may adopt the detector 3I shown in FIG. 17 instead of the detector 3H.

Specifically, in the detector 3I, the light emitters 321 and 322 are disposed on the mounting surface 311 to differentiate a distance L61 between the first light emitter 321 and the outer surface 3412 of the side portion 341 and a distance L62 between the second light emitter 322 and the outer surface 3422 of the side portion 342.

Note that, in the second modification, the distance L61 is set smaller than the distance L62. However, the invention is not limited to this. For example, the distance L61 may be larger than the distance L62. That is, the distance L61 and the distance L62 only have to be different distances.

That is, since the distance L61 and the distance L62 are different, as explained above, it is possible to surely differentiate the lengths of the optical paths or the routes of the light emitted from the first light emitter 321 and made incident on the first light receiver 331 and the second light receiver 332 via the detection part. Therefore, in the second modification, it is possible to achieve effects same as the effects of the third embodiment.

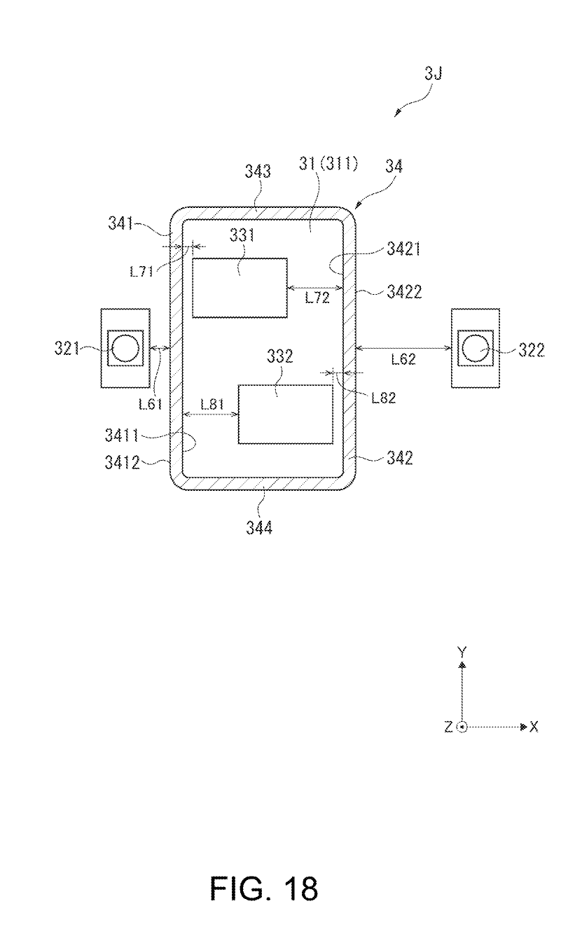

FIG. 18 is a plan view of a detector 3J in a third modification of this embodiment viewed from the back section 11B side.

In the second modification, in the first light receiver 331 and the second light receiver 332, the distances L11, L12, L21, and L22 from the inner surfaces 3411 and 3421 of both the side portions 341 and 342 of the frame 34 are the same. However, the invention is not limited to this. For example, the biological information detection device may adopt the detector 3J shown in FIG. 18 instead of the detector 3I.

Specifically, in the detector 3J, the first light receiver 331 is disposed in a position on the opposite side of the X direction on the mounting surface 311 of the substrate 31 and the second light receiver 332 is disposed in a position on the X-direction side on the mounting surface 311 of the substrate 31. Therefore, a distance L71 from the first light receiver 331 to the inner surface 3411 of the side portion 341 and a distance L72 from the first light receiver 331 to the inner surface 3421 of the side portion 342 are different. A distance L81 from the second light receiver 332 to the inner surface 3411 and a distance L82 from the second light receiver 332 to the inner surface 3421 are different.

That is, since the distances L71 and L81 and the distances L72 and L82 are different, as in the second modification, lengths of optical paths or routes of the light emitted from the first light emitter 321, reflected in the wearing part (the detection part), and made incident on the light receivers 331 and 332 are different from each other and lengths of optical paths or routes of the light emitted from the second light emitter 322, reflected in the wearing part (the detection part), and made incident on the light receivers 331 and 332 are different from each other. Therefore, in the third modification, it is possible to achieve effects same as the effects of the third embodiment.

### Fourth Embodiment

A fourth embodiment of the invention is explained.

A biological information detection device according to this embodiment includes a configuration same as the configuration of the biological information detection device 1. However, the configuration of the detector 3 is different. That is, in this embodiment, the detector 3 includes a frame having a shape different from the shape of the frame 34. In the first embodiment, the detector 3 includes the pedestal 35 on the mounting surface 311 of the substrate 31. However, in this embodiment, the detector 3 does not include the pedestal 35. The biological information detection device according to this embodiment and the biological information detection device 1 are different in these points. Note that, in the following explanation, portions the same as or substantially the same as the portions already explained above are denoted by the same reference numerals and signs and explanation of the portions is omitted.

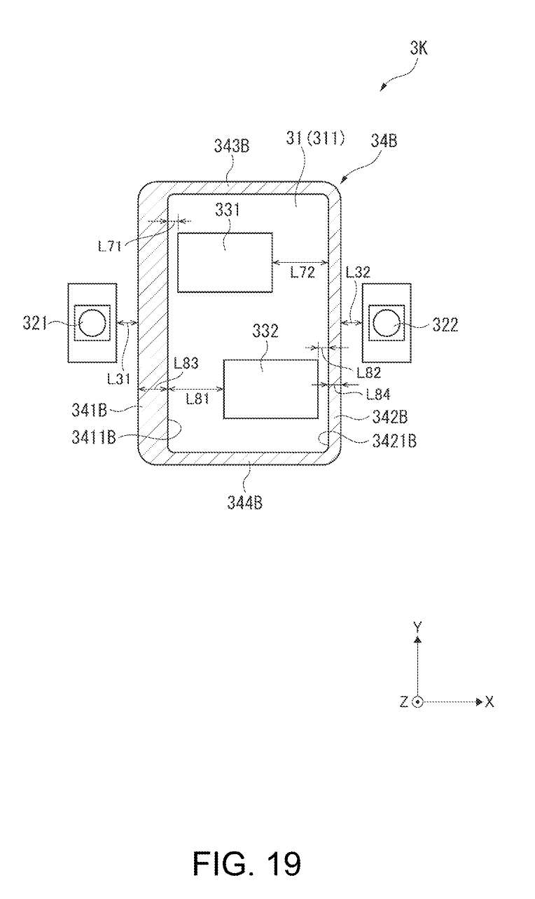

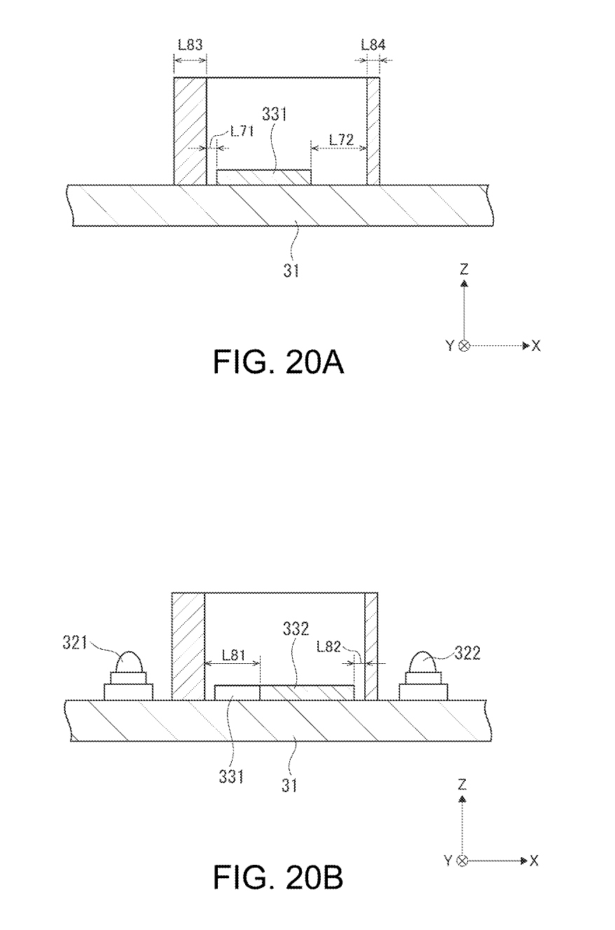

FIG. 19 is a plan view of a detector 3K in this embodiment viewed from the back section 11B side. FIG. 20A is a sectional view showing a sectional shape perpendicular to a frame 34B including the center of the first light receiver 331. FIG. 20B is a sectional view showing a sectional shape perpendicular to the frame 34B including the center of the second light receiver 332.

The biological information detection device according to this embodiment includes components and functions same as those of the biological information detection device 1 except that the biological information detection device includes the detector 3K instead of the detector 3. As shown in FIG. 19, the detector 3K includes the frame 34B instead of the frame 34. In the frame 34B, a thickness dimension L83 of a side portion 341B on the first light receiver 331 side is formed larger than a thickness dimension L84 of a side portion 342B on the second light receiver 332 side. That is, as shown in FIGS. 20A and 20B, a sectional shape on the first light receiver 331 side in the frame 34B and a sectional shape on the second light receiver 332 side in the frame 34B are different. The distances L71, L72, L81, and L82 of the first light receiver 331 and the second light receiver 332 to the frame 34B are different. In other words, a sectional shape perpendicular to the frame 34B including the center of the first light receiver 331 and a sectional shape perpendicular to the frame 34B including the center of the second light receiver 332 are different.

As in the third modification of the third embodiment, the first light receiver 331 and the second light receiver 332 are disposed on the mounting surface 311 of the substrate 31. Therefore, the distance L71 from the first light receiver 331 to an inner surface 3411B of the side portion 341B and the distance L72 from the first light receiver 331 to an inner surface 3421B of the side portion 342B are different. The distance L81 from the second light receiver 332 to the inner surface 3411B and the distance L82 from the second light receiver 332 to the inner surface 3421B are different.

That is, the distances L71 and L81 and the distances L72 and L82 are different and the thickness dimension of the side portion 341B and the thickness dimension (the sectional shape) of the side portion 342B are different. Therefore, lengths of optical paths or routes of light emitted from the first light emitter 321, reflected in the wearing part (the detection part), and made incident on the light receivers 331 and 332 are different from each other. Lengths of optical paths or routes of the light emitted from the second light emitter 322, reflected in the wearing part (the detection part), and made incident on the light receivers 331 and 332 are different from each other.

Note that, in this embodiment, cross sections perpendicular to the frame 34B including the centers of first light receiver 331 and the second light receiver 332 are different from each other. However, the invention is not limited to this. For example, a sectional shape passing the centers of the light emitters 321 and 322 and the center of the first light receiver 331 and a sectional shape passing the centers of the light emitters 321 and 322 and the center of the second light receiver 332 may be different.

### Effects of the Fourth Embodiment

The biological information detection device according to this embodiment explained above has effects explained below besides effects same as the effects of the biological information detection device 1 in the embodiments.

In this embodiment, the sectional shapes perpendicular to the frame 34B including the centers of the first light receiver 331 and the second light receiver 332 are different from each other. Therefore, as in the first embodiment, it is possible to surely differentiate the lengths of the optical paths or the routes of the light including the different kinds of information emitted from the first light emitter 321 and made incident on the first light receiver 331 and the second light receiver 332 via the detection part. In other words, the first light receiver 331 and the second light receiver 332 can receive the lights having the different kinds of information. Therefore, since it is possible to surely differentiate light reception results by the first light receiver 331 and the second light receiver 332, it is possible to surely improve the detection and analysis accuracy of biological information.

The frame 34B in which a part on the first light receiver 331 side (the side portion 341B) and a part on the second light receiver 332 side (the side portion 342B) are different is provided. Therefore, it is unnecessary to provide the pedestal 35. Therefore, it is possible to reduce manufacturing costs and manufacturing expenses of the detector 3K. Further, it is possible to reduce manufacturing costs and manufacturing expenses of the biological information detection device.

### Modifications of the Fourth Embodiment

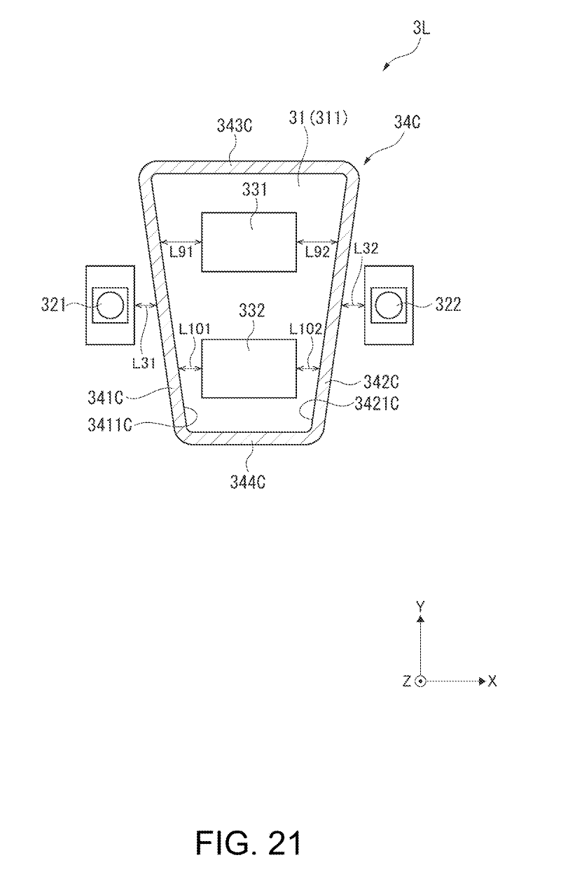

FIG. 21 a plan view of a detector 3L according to a first modification of the fourth embodiment viewed from the back section 11B side.

In the fourth embodiment, the thickness dimension L83 of the side portion 341B on the first light emitter 321 side and the thickness dimension L84 of the side portion 342B on the second light emitter 322 side of the frame 34B are different. The light receivers 331 and 332 are disposed on the mounting surface 311 of the substrate 31 in positions same as the positions in the third modification of the third embodiment. However, the invention is not limited to this. For example, the biological information detection device may adopt the detector 3L shown in FIG. 21 instead of the detector 3K.

Specifically, in the detector 3L, the light receivers 331 and 332 are disposed on the mounting surface 311 of the substrate 31. The detector 3L includes a frame 34C instead of the frame 34B.

As shown in FIG. 21, the frame 34C is a cylindrical body, the cross section in an XY direction of which is a trapezoidal shape. Specifically, the length in the X direction of the side portion 343C is set larger than the length in the X direction of the side portion 344C. Consequently, distances L91 and L92 from the first light receiver 331 to inner surfaces 3411C and 3421C of side portions 341C and 342C are larger than distances L101 and L102 from the second light receiver 332 to inner surfaces 3411C and 3421C of the side portions 341C and 342C.

That is, the shape on the first light receiver 331 side in the frame 34C and the shape on the second light receiver 332 side in the frame 34C are different and the distances L91 and L92 and the distances L101 and L102 are different. Therefore, lengths of optical paths or routes of the light emitted from the first light emitter 321, reflected in the wearing part (the detection part), and made incident on the light receivers 331 and 332 are different from each other and lengths of optical paths or routes of the light emitted from the second light emitter 322, reflected in the wearing part (the detection part), and made incident on the light receivers 331 and 332 are different from each other. Therefore, in the first modification, it is possible to achieve effects same as the effects of the fourth embodiment.

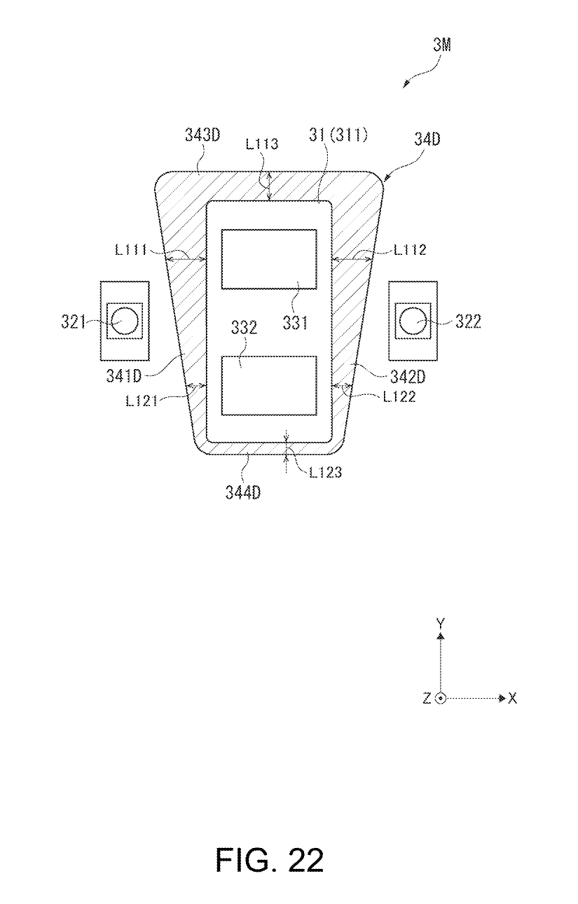

FIG. 22 is a plan view of a detector 3M according to a second modification of this embodiment viewed from the back section 11B side.

In the first modification of the fourth embodiment, the thickness dimensions of the side portions 341C to 344C of the frame 34C are the same. However, the invention is not limited to this. For example, the biological information detection device may adopt the detector 3M shown in FIG. 22 instead of the detector 3L.

Specifically, the detector 3M includes a frame 34D instead of the frame 34C. The frame 34D is a cylindrical body, the cross section in the XY direction of which is a trapezoidal shape. The detector 3M includes side portions 341D to 344D. The thickness dimensions of the side portions 341D to 344D are different from one another. Among the side portions 341D to 344D, in the side portions 341D and 342D, the thickness dimensions decreases from the Y direction toward the opposite direction of the Y direction. Therefore, thickness dimension L111 and L112 in the vicinity of the first light receiver 331 in the side portions 341D and 342D are large compared with thickness dimensions L121 and L122 in the vicinity of the second light receiver 332. A thickness dimension L113 of the side portion 343D located on the first light receiver 331 side is large compared with a thickness dimension L123 of the side portion 344D located on the second light receiver 332 side.

That is, the shape (the sectional shape) on the first light receiver 331 side in the frame 34D and the shape (the sectional shape) on the second light receiver 332 side in the frame 34D are different and the thickness dimensions L111 to L113 and the thickness dimensions L121 to L123 are different. Therefore, as in the first modification, lengths of optical paths or routes of the light emitted from the first light emitter 321, reflected in the wearing part (the detection part), and made incident on the light receivers 331 and 332 are different from each other and lengths of optical paths or routes of the light emitted from the second light emitter 322, reflected in the wearing part (the detection part), and made incident on the light receivers 331 and 332 are different from each other. Therefore, in the second modification, it is possible to achieve effects same as the effects of the fourth embodiment.

### Fifth Embodiment

A fifth embodiment of the invention is explained below.

A biological information detection device according to this embodiment includes a configuration same as the configuration of the biological information detection device 1. However, the configuration of the detector 3 is different. That is, in this embodiment, as in the first embodiment, the detector 3 includes the substrate 31, the light emitters 321 and 322, the light receivers 331 and 332, the frame 34, and the pedestal 35. However, the disposition of the light emitters 321 and 322, the light receivers 331 and 332, the frame 34, and the pedestal 35 on the mounting surface 311 of the substrate 31 is different. The biological information detection device according to this embodiment and the biological information detection device 1 are different in this point. Note that, in the following explanation, portions the same as or substantially the same as the portions already explained above are denoted by the same reference numerals and signs and explanation of the portions is omitted.

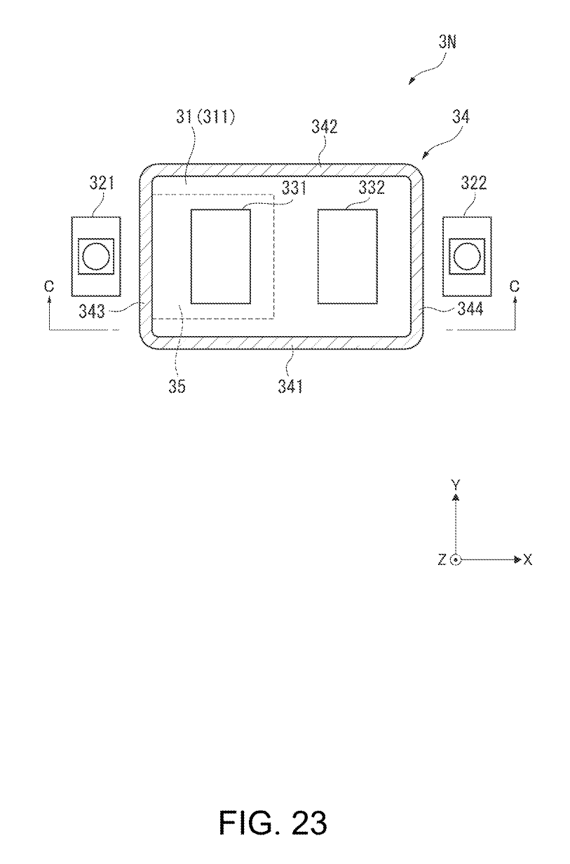

FIG. 23 is a plan view of a detector 3N of the biological information detection device according to this embodiment viewed from the back section 11B side.

The biological information detection device according to this embodiment includes components and functions same as those of the biological information detection device 1 except that the biological information detection device includes the detector 3N instead of the detector 3. The detector 3N includes, as shown in FIG. 23, the substrate 31, the light emitters 321 and 322, the light receivers 331 and 332, the frame 34, and the pedestal 35. On the mounting surface 311 of the substrate 31, the light receivers 331 and 332 and the frame 34 are disposed in a state in which the light receivers 331 and 332 and the frame 34 are rotated 90° from those in the biological information detection device 1. Specifically, when viewed from the Z direction, the side portion 342 of the frame 34 is disposed to be located most distant in the Y direction and the side portion 341 of the frame 34 is located most distant in the opposite direction of the Y direction. In a region of the frame 34, that is, in a region in the center of the mounting surface 311, the first light receiver 331 and the second light receiver 332 are disposed side by side along the X direction. Specifically, the first light receiver 331 is disposed on the side portion 343 side of the frame 34 and the second light receiver 332 is disposed on the side portion 344 side of the frame 34.

On the outer side of the frame 34 on the mounting surface 311, the first light emitter 321 and the second light emitter 322 are disposed along the X direction. Specifically, the first light emitter 321 is disposed on the opposite direction side of the X direction of the side portion 343 of the frame 34 on the mounting surface 311 and the second light emitter 322 is disposed on the X-direction side of the side portion 344 of the frame 34 on the mounting surface 311.

That is, the light receivers 331 and 332 and the light emitters 321 and 322 are disposed in the same linear shape extending along the X direction.

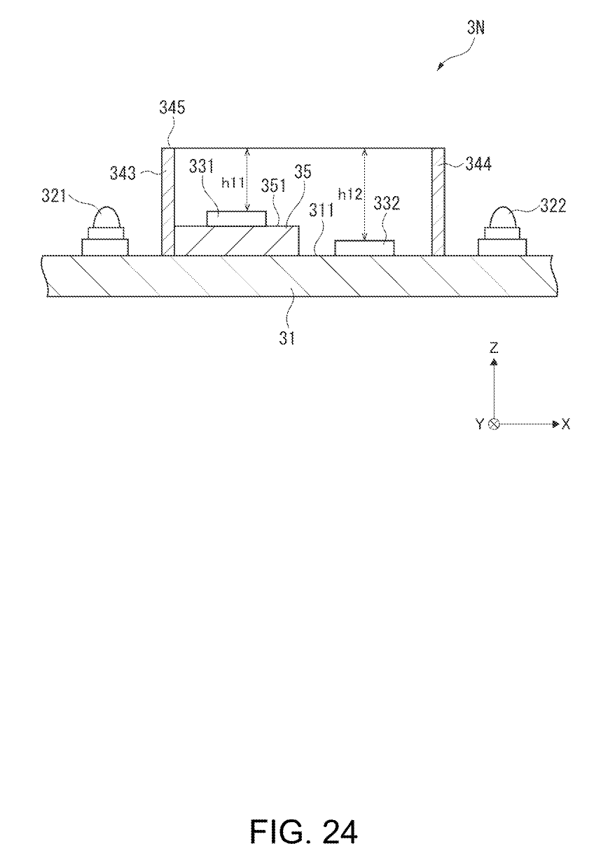

FIG. 24 is a sectional view of a C-C section in FIG. 23 in the detector 3N viewed from the opposite side of the Y direction (the vertical direction of the straight line connecting the center of the first light emitter 321 and the center of the second light emitter 322).

On the mounting surface 311 of the substrate 31, as shown in FIG. 24, the rectangular plate-like pedestal 35 is disposed. On the upper surface 351 of the pedestal 35, the first light receiver 331 is disposed. Consequently, the distance h11 from the first light receiver 331 to the upper end face 345 of the frame 34 is shorter than the distance h12 from the second light receiver 332 to the upper end face 345. In other words, the first light receiver 331 and the second light receiver 332 are respectively disposed in positions at different distances from the mounting surface 311 of the substrate 31.

That is, in this embodiment as well, the first light receiver 331 and the second light receiver 332 are respectively disposed in positions on the mounting surface 311 where lengths of optical paths or routes of the light emitted from the first light emitter 321, reflected in the wearing part (the detection part), and made incident on the light receivers 331 and 332 are different from each other and lengths of optical paths or routes of the light emitted from the second light emitter 322, reflected in the wearing part (the detection part), and made incident on the light receivers 331 and 332 are different from each other.

Note that, in FIG. 24, the upper surface of the first light emitter 321 and the upper surface of the second light emitter 322 further project to the Z-direction side than the upper surface of the first light receiver 331 or the upper surface of the second light receiver 332. That is, the distance between the substrate 31 and the upper surfaces of the light emitters 32 is depicted as being longer than the distance between the substrate 31 and the upper surfaces of the light receivers 33. However, the invention is not limited to this. For example, the upper surface of the first light receiver 331 and the upper surface of the second light receiver 332 may be configured to further project in the Z direction than the upper surface of the first light emitter 321 or the upper surface of the second light emitter 322. That is, the distance between the substrate 31 and the light receiving surfaces of the light receivers 33 may be set longer than the distance between the substrate 31 and the upper surfaces of the light emitters 32. However, the upper end face 345 of the frame 34 is configured to further project in the Z-direction side than the upper surfaces of the light emitters 32 and the upper surfaces of the light receivers 33. With such a configuration, it is possible to differentiate the lengths of the optical paths or the routes of the light emitted from the first light emitter 321 and the light emitted from the second light emitter 322 while applying an appropriate pressing force to the light receiving surfaces. It is possible to improve the detection and analysis accuracy of biological information.

### Effects of the fifth embodiment

The biological information detection device according to this embodiment explained above has effects explained below besides effects same as the effects of the biological information detection device 1 in the embodiments.

In this embodiment, as in the first embodiment, it is possible to surely differentiate the lengths of the optical paths or the routes of the light including the different kinds of information emitted from the first light emitter 321 and made incident on the first light receiver 331 and the second light receiver 332 via the detection part. In other words, the first light receiver 331 and the second light receiver 332 can receive the lights having the different kinds of information. Therefore, since it is possible to surely differentiate light reception results by the first light receiver 331 and the second light receiver 332, it is possible to surely improve the detection and analysis accuracy of biological information.

The light emitters 321 and 322 and the light receivers 331 and 332 are disposed on the straight line extending along the X direction. Therefore, it is possible to reduce a space extending along the Y direction of the detector 3N.

### Modification of the Fifth Embodiment

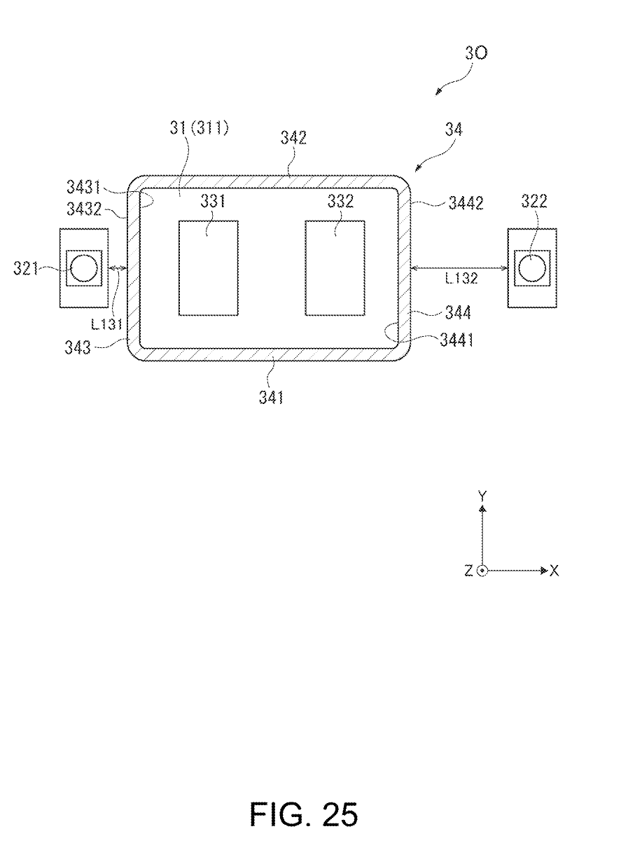

FIG. 25 is a plan view of a detector 3O in a modification of this embodiment viewed from the back section 11B side.

In the fifth embodiment, the first light emitter 321 is disposed on the upper surface 351 of the pedestal 35. However, the invention is not limited to this. For example, the biological information detection device may adopt the detector 3O shown in FIG. 25 instead of the detector 3N.

Specifically, in the detector 3O, the light emitters 321 and 322 may be disposed on the mounting surface 311 to differentiate a distance L131 between the first light emitter 321 and an outer surface 3432 of the side portion 343 and a distance L132 between the second light emitter 322 and an outer surface 3442 of the side portion 344.

Note that, in the modification, the distance L131 is set smaller than the distance L132. However, the invention is not limited to this. For example, the distance L131 may be larger than the distance L132. That is, the distance L131 and the distance L132 only have to be different distances.

In this way, since the distance L131 and the distance L132 are different, as in the fifth embodiment, lengths of optical paths or routes of the light emitted from the first light emitter 321, reflected in the wearing part (the detection part), and made incident on the first light receiver 331 and the second light receiver 332 are different from each other and lengths of optical paths or routes of the light emitted from the second light emitter 322, reflected in the wearing part (the detection part), and made incident on the light receivers 331 and 332 are different from each other. Therefore, in the modification, it is possible to achieve effects same as the effects of the fifth embodiment.

### Sixth Embodiment

A sixth embodiment of the invention is explained.

A biological information detection device according to this embodiment includes a configuration same as the configuration of the biological information detection device 1. However, the configuration of the detector 3 is different. That is, in this embodiment, as in the fifth embodiment, the detector 3 includes the substrate 31, the first light emitter 321, the light receivers 331 and 332, and the frame 34. However, the detector 3 does not include the pedestal 35 and the second light emitter 322. The biological information detection device according to this embodiment and the biological information detection device 1 are different in this point. Note that, in the following explanation, portions the same as or substantially the same as the portions already explained above are denoted by the same reference numerals and signs and explanation of the portions is omitted.

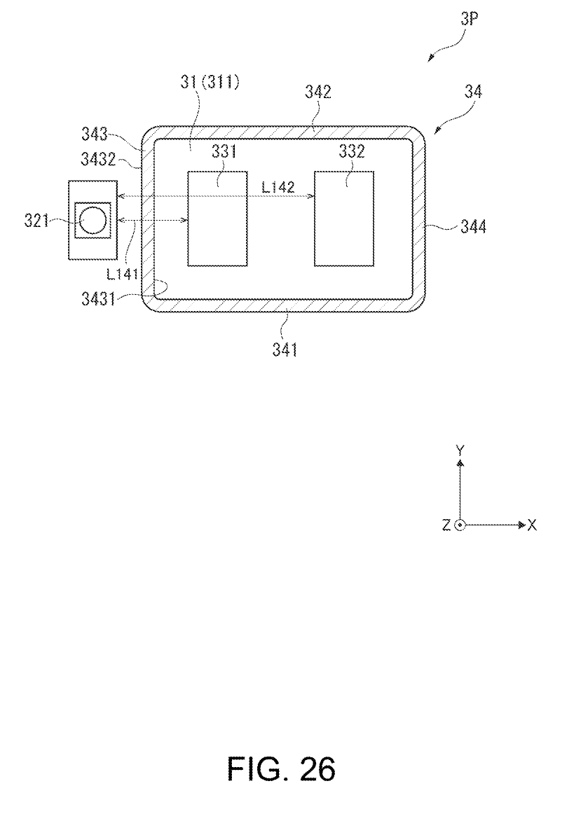

FIG. 26 is a plan view of a detector 3P of the biological information detection device according to this embodiment viewed from the back section 11B.

The biological information detection device according to this embodiment includes components and functions same as those of the biological information detection device 1 except that the biological information detection device includes the detector 3P instead of the detector 3. The detector 3P includes, as shown in FIG. 26, the substrate 31, the light emitter 321, the light receivers 331 and 332, and the frame 34. The first light receiver 331 and the second light receiver 332 are disposed side by side along the X direction in the region in the center of the mounting surface 311. The first light emitter 321 is disposed on the outer side of the frame 34 on the mounting surface 311. Specifically, the first light emitter 321 is disposed on the opposite direction side of the X direction of the side portion 343 of the frame 34 on the mounting surface 311. That is, the light receivers 331 and 332 and the first light emitter 321 are disposed in a same linear shape extending along the X direction.

Therefore, a distance L141 from the first light emitter 321 to the first light receiver 331 is shorter than a distance L142 from the first light emitter 321 to the second light receiver 332. That is, since the distance L141 and the distance L142 are different, lengths of optical paths or routes of the light emitted from the first light emitter 321, reflected in the wearing part (the detection part), and made incident on the light receivers 331 and 332 are different from each other.

### Effects of the Sixth Embodiment

The biological information detection device according to this embodiment explained above has effects explained below besides effects same as the effects of the biological information detection device 1 in the embodiments.

In this embodiment, the distance L141 from the first light receiver 331 to the first light emitter 321 and the distance L142 from the second light receiver 332 to the first light emitter 321 are different. Therefore, it is possible to surely differentiate the lengths of the optical paths or the routes of the light including the different kinds of information emitted from the first light emitter 321 and made incident on the first light receiver 331 and the second light receiver 332 via the detection part. In other words, the first light receiver 331 and the second light receiver 332 can receive the lights having the different kinds of information. Therefore, since it is possible to surely differentiate light reception results by the first light receiver 331 and the second light receiver 332, it is possible to surely improve the detection and analysis accuracy of biological information.

In this embodiment, it is unnecessary to provide the second light emitter 322 provided in the other embodiments. Therefore, it is possible to reduce the detector 3P in size. Further, it is possible to reduce manufacturing costs and manufacturing expenses of the biological information detection device.

### Seventh Embodiment

A seventh embodiment of the invention is explained.

The biological information detection device according to this embodiment includes a configuration same as the configuration of the biological information detection device 1. However, the configuration of the detector 3 is different. That is, in this embodiment, the detector 3 includes the substrate 31, the light emitters 321 and 322, and the light receivers 331 and 332. However, the detector 3 does not include the pedestal 35. In this embodiment, the detector 3 includes a frame 34E instead of the frame 34. Further, in this embodiment, the detector 3 includes a resin layer. The biological information detection device according to this embodiment is different from the biological information detection device 1 in these points. Note that, in the following explanation, portions the same as or substantially the same as the portions already explained above are denoted by the same reference numerals and signs and explanation of the portions is omitted.

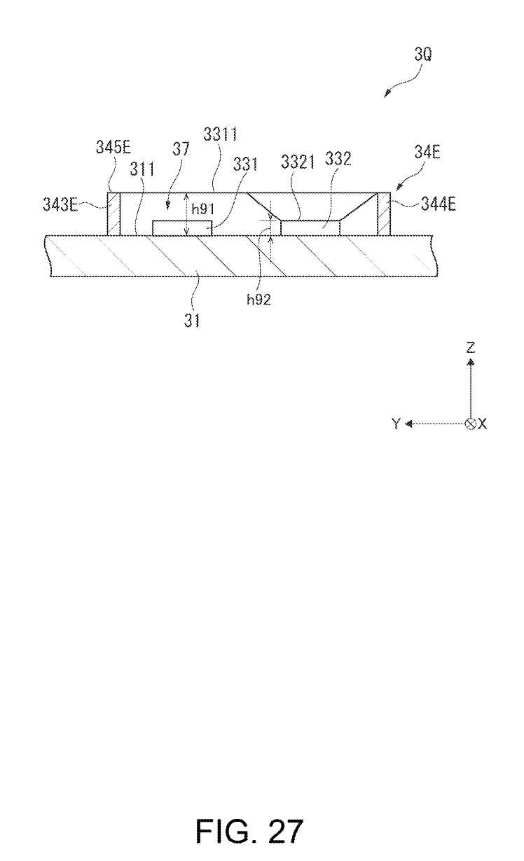

FIG. 27 is a sectional view of the A-A section in FIG. 4 in a detector 3Q of the biological information detection device according to this embodiment viewed from the right side section 11R side.

The biological information detection device according to this embodiment includes components and functions same as those of the biological information detection device 1 except that the biological information detection device includes the detector 3Q instead of the detector 3. The detector 3Q includes, as shown in FIG. 23, the substrate 31, the light emitters 321 and 322, the light receivers 331 and 332, the frame 34, and a resin layer 37.

The frame 34E includes a configuration substantially the same as the configuration of the frame 34. However, height in the Z direction is set small compared with the frame 34.

The resin layer 37 is equivalent to the transparent member of the invention and is formed of light transmissive resin having a function of transmitting light. As shown in FIG. 27, the resin layer 37 is disposed in a range surrounded by the frame 34E on the mounting surface 311 of the substrate 31. Specifically, the resin layer 37 is filled in the range to cover the first light receiver 331 of the light receivers 331 and 332. Specifically, on the first light receiver 331 side, the resin layer 37 is filled up to an upper end portion 345E of the frame 34 and, on the second light receiver 332 side, the resin layer 37 is filled to avoid the upper surface of the second light receiver 332. In this way, the resin layer 37 is disposed around the first light receiver 331 and the second light receiver 332. Therefore, the second light receiver 332 is not covered by the resin layer 37. A distance h91 from the substrate 31 to a light receiving surface 3311 of the first light receiver 331 is shorter than a distance h92 from the substrate 31 to a light receiving surface 3321 of the second light receiver 332. That is, the light receiving surface 3311 of the first light receiver 331 and the light receiving surface 3321 of the second light receiver 332 are respectively disposed in positions at different distances from the substrate 31.

Note that, in this embodiment, the resin layer 37 is formed of the light transmissive resin. However, the invention is not limited to this. For example, the resin layer 37 may be formed of, for example, glass or the like having light transmissivity. That is, the resin layer 37 may be formed of any member as long as the member has light transmissivity.

As explained above, the first light receiver 331 is covered by the resin layer 37 and the second light receiver 332 is not covered by the resin layer 37. Therefore, a thickness dimension of the resin layer 37 on the first light receiver 331 side is larger than a thickness dimension of the resin layer 37 on the second light receiver 332 side. Consequently, when the biological information detection device according to this embodiment is worn on the user, pressing forces applied to the detector 3Q from the detection part of the user are different. More specifically, a pressing force applied to the first light receiver 331 from the detection part and a pressing force applied to the second light receiver 332 from the detection part are different.

### Effects of the Seventh Embodiment

The biological information detection device according to this embodiment explained above has effects explained below besides effects same as the effects of the biological information detection device 1 in the embodiments.

According to this embodiment, when the biological information detection device is worn on the user, it is possible to differentiate, with the resin layer 37 functioning as the pressing force adjuster, a pressing force on the first light receiver 331 side applied to the detection part and a pressing force on the second light receiver 332 side applied to the detection part. Consequently, it is possible to differentiate an incident angle of light made incident on the first light receiver 331 via the detection part and an incident angle of light made incident on the second light receiver 332 via the detection part. Therefore, since it is possible to surely differentiate light reception results by the first light receiver 331 and the second light receiver 332, it is possible to surely improve the detection and analysis accuracy of biological information.

The light receiving surface 3311 of the first light receiver 331 and the light receiving surface 3321 of the second light receiver 332 are respectively disposed in the positions at the different distances from the substrate 31. Therefore, it is possible to differentiate the lengths of the optical paths or the routes of the light emitted from the first light emitter 321 and made incident on the first light receiver 331 and the second light receiver 332 via the detection part. In other words, the first light receiver 331 and the second light receiver 332 can receive the lights having the different kinds of information. Therefore, since it is possible to differentiate light reception results by the first light receiver 331 and the second light receiver 332, it is possible to improve the detection and analysis accuracy of biological information.

### Modifications of the Embodiments

The invention is not limited to the embodiments. Modifications, improvements, and the like in a range in which the object of the invention can be achieved are included in the invention.

In the embodiments, the disposition of the light emitters 321 and 322, the light receivers 331 and 332, and the frames 34 and 34A to 34E on the mounting surface 311 of the substrate 31 explained in the embodiments can be changed as appropriate. That is, the light emitters 321 and 322, the light receivers 331 and 332, and the frames 34 and 34A to 34E may be disposed on the mounting surface 311 any way as long as lengths of optical paths of the light emitted from the first light emitter 321, reflected in the wearing part (the detection part), and received by the light receivers 331 and 332 are different from each other and lengths of optical paths of the light emitted from the second light emitter 322, reflected in the wearing part (the detection part), and received by the light receivers 331 and 332 are different from each other.

In the embodiments, the frames 34 and 34A to 34E are the wall-like convex structures formed in the substantially cylindrical shape and extending in the Z direction from the substrate 31. However, the invention is not limited to this. For example, the frames 34 and 34A to 34E may be configured by a plurality of plate-like bodies and disposed between the first light emitter 321 and the first light receiver 331 and between the second light emitter 322 and the second light receiver 332. That is, the frames 34 and 34A to 34E may have any configuration as long as the frames 34 and 34A to 34E can block lights, which are emitted from the light emitters 321 and 322, made directly incident on the light receivers 331 and 332.

In the embodiments, the light receivers 33 include the first light receiver 331 and the second light receiver 332. However, the invention is not limited to this. For example, in addition to the light receivers 331 and 332, one or a plurality of light receivers may be provided.

In the embodiments, the light receivers 331 and 332 are respectively disposed on the substrate 31 to differentiate lengths of optical paths of the lights emitted from the light emitters 32 (the first and second light emitters 321 and 322 or the first light emitter 321). However, the invention is not limited to this. For example, the light receivers 331 and 332 may be respectively disposed on the substrate 31 to equalize lengths of optical paths of the lights emitted from the light emitters 32.

In the first to sixth embodiments, the resin layer 37 is not disposed in the frames 34 and 34A to 34D. However, the invention is not limited to this. For example, in the frames 34 and 34A to 34D in the first to sixth embodiments, the resin layer 37 may be disposed. Note that, in these cases, the resin layer 37 may be filed to reach the upper end faces 345 and 345A to 345D of the frames 34 and 34A to 34D. Further, the resin layer 37 may project beyond the upper end faces 345 and 345A to 345D of the frames 34 and 34A to 34D. That is, the light receivers 331 and 332 may be covered by the resin layer 37. Further, as in the seventh embodiment, the first light receiver 331 may be covered by the resin layer 37 and the second light receiver 332 may not be covered by the resin layer 37.

In the above case and the seventh embodiment, the first light receiver 331 may not be covered by the resin layer 37 and the second light receiver 332 may be covered by the resin layer 37. Further, both of the light receivers 331 and 332 may not be covered by the resin layer 37. That is, the resin layer 37 may be disposed in any way as long as a pressing force on the first light receiver 331 side and a pressing force on the second light receiver 332 side are different when the detection device 1 is worn.

In the first to third and fifth embodiments, the pedestals 35, 35A, 35B, and 36 are disposed on the mounting surface 311 of the substrate 31. However, the invention is not limited to this. For example, the pedestals 35, 35A, 35B, and 36 may be formed integrally with the substrate 31.

In the embodiments, the detection device 1 includes the acceleration sensor 14 as the body motion information detector. However, the invention is not limited to this. For example, in the embodiments, the detection device 1 may not include the acceleration sensor 14. In this case, the detection device 1 only has to detect biological information (a pulse wave) on the basis of only the first and second detection signals detected by the light receivers 331 and 332.

In the embodiments, the detection device 1 is formed in a wristwatch shape. The wearing part of the detection device 1 is the wrist of the wearer. However, the invention is not limited to this. For example, the detection device 1 may be worn on an ankle, the chest, and the abdomen of the wearer.

What is claimed is:

1. A biological information detection sensor comprising:
a first light emitter configured to emit light to a detection part;
a first light receiver and a second light receiver configured to receive the light reflected in the detection part;
a light blocker disposed between the first light emitter and the first and second light receivers and configured to block the light made directly incident on the first light receiver and the second light receiver from the first light emitter;
a supporting body on which the first light emitter, the first light receiver, the second light receiver, and the light blocker are disposed; and
a controller configured to analyze biological information on the basis of light reception result received from the first light receiver and the second light receiver.

2. The biological information detection sensor according to claim 1, wherein the light blocker configures at least apart of a frame that surrounds the first light receiver and the second light receiver viewed from the detection part side.

3. The biological information detection sensor according to claim 1, wherein the first light emitter and the first and second light receivers are disposed to differentiate a distance between the first light emitter and the first light receiver and a distance between the first light emitter and the second light receiver.

4. The biological information detection sensor according to claim 1, wherein a light receiving surface of the first light receiver and a light receiving surface of the second light receiver are respectively disposed in positions at different distances from the supporting body.

5. The biological information detection sensor according to claim 1, wherein an upper surface of the first light receiver and an upper surface of the second light receiver are respectively disposed in positions at different distances from the supporting body.

6. The biological information detection sensor according to claim 1, wherein a sectional shape including a center of the first light receiver and perpendicular to the light blocker and a sectional shape including a center of the second light receiver and perpendicular to the light blocker are different.

7. The biological information detection sensor according to claim 1, wherein a distance between the light blocker and the first light receiver and a distance between the light blocker and the second light receiver are different in plan view.

8. The biological information detection sensor according to claim 1, wherein a distance from the supporting body to an upper surface of the light blocker is longer than a distance from the supporting body to the light receiving surface of the first light receiver and the light receiving surface of the second light receiver in plan view.

9. The biological information detection sensor according to claim 1, further comprising a second light emitter disposed on the supporting body and configured to emit light to the detection part, wherein
the first light emitter and the second light emitter are disposed in positions where at least one of the distances from the supporting body, the distances from the first light receiver, and the distances from the second light receiver are different.

10. The biological information detection sensor according to claim 1, further comprising a transparent member disposed around at least one of the first light receiver and the second light receiver and configured to differentiate, on the first light receiver side and the second light receiver side, pressing forces applied to the detection part.

11. A biological information detection device comprising the biological information detection sensor according to claim 1.

12. A biological information detection device comprising the biological information detection sensor according to claim 2.

13. A biological information detection device comprising the biological information detection sensor according to claim 3.

14. A biological information detection device comprising the biological information detection sensor according to claim 4.

15. A biological information detection device comprising the biological information detection sensor according to claim 5.

16. A biological information detection device comprising the biological information detection sensor according to claim 6.

17. A biological information detection device comprising the biological information detection sensor according to claim 7.

18. A biological information detection device comprising the biological information detection sensor according to claim 8.

19. A biological information detection device comprising the biological information detection sensor according to claim 9.

20. A biological information detection device comprising the biological information detection sensor according to claim 10.
